(19) United States
(12) Patent Application Publication
(10) Pub. No.: US 2026/0358323 A1
(43) Pub. Date: Nov. 12, 2026

(54) SOIL SENSING SYSTEM AND METHOD

(71) Applicant: Yale University, New Haven, CT (US)

(72) Inventors: Jian Ding, New Haven, CT (US); Leandros Tassiulas, New Haven, CT (US)

(21) Appl. No.: 19/671,741

(22) Filed: May 8, 2026

Related U.S. Application Data
(60) Provisional application No. 63/802,959, filed on May 9, 2025.

(51) Int. Cl. G01N 33/24

(52) U.S. Cl. CPC G01N 33/246; G01N 33/243

(57) ABSTRACT

A method for detecting soil carbon content is described. The method includes providing an image of a soil surface, determining soil lightness of the soil surface based on the image, providing channel state information (CSI) of the soil, estimating carbon content of the soil based on the soil lightness and the CSI, the estimation being based on a mathematical model, refining the estimated carbon content, the refinement comprising predicting an error between the estimated carbon content and the true carbon content using a machine learning model, and adding the error to the estimated carbon content.

### CROSS-REFERENCE TO RELATED APPLICATIONS

This application claims priority to U.S. Provisional Application No. 63/802,959, filed on May 9, 2025, which is incorporated herein by reference in its entirety.

### STATEMENT REGARDING FEDERALLY SPONSORED RESEARCH OR DEVELOPMENT

This invention was made with government support under W911NF-23-1-0088 awarded by the Army Research Office and 2132573 awarded by the National Science Foundation. The government has certain rights in the invention.

### BACKGROUND OF THE INVENTION

Soil carbon is a critical factor in maintaining soil health and combating climate change. Understanding and managing soil carbon levels is essential for sustainable agriculture and environmental protection. However, current methods for measuring soil carbon are time-consuming and costly, hindering efforts to monitor soil health and increase carbon sequestration.

There remains a need in the art for methods for measuring soil health.

### SUMMARY OF THE INVENTION

In some aspects, the invention relates to a method for detecting soil carbon content.

In some embodiments, the method comprises the steps of providing an image of a soil surface, determining soil lightness of the soil surface based on the image, providing channel state information (CSI) of the soil, estimating carbon content of the soil based on the soil lightness and the CSI wherein the estimation of carbon content is based on a mathematical model, refining the estimated carbon content, the refinement comprising predicting an error between the estimated carbon content and the true carbon content using a machine learning model, and adding the predicted error to the estimated carbon content.

In some embodiments, the method further comprises the steps of estimating the soil water content based on the soil lightness and the CSI wherein the estimation of soil lightness is based on a mathematical model, refining the estimated soil water content comprising predicting an error between the estimated soil water content and the true soil water content using a machine learning model, and adding the predicted error to the estimated soil water content.

In some embodiments, the machine learning model of the method is trained on images of soil surfaces, soil CSI, and ground truth carbon content.

In some embodiments, estimating the carbon content of the soil comprises determining the effective permittivity of the soil based on the soil CSI.

In some embodiments, the soil CSI is determined via assessing the propagation of a radiofrequency (RF) signal in the soil, wherein the RF signal comprises an RF signal selected from the group consisting of: a Wi-Fi signal, a radiofrequency identification (RFID) signal, a backscatter signal, a Radar signal, a LoRa signal, an ultra-wide-band signal, and a long-term evolution (LTE) signal.

In some embodiments, the image of a soil surface comprises a smartphone image.

In some embodiments, propagated RF signal for determining CSI is propagated between two RF nodes wherein at least one of the RF nodes is buried in the soil.

In some embodiments, the machine learning model of the method comprises a convolutional neural network (CNN).

In some embodiments, the CNN comprises a CNN selected from the group consisting of: ResNet, VGGNet, Inception, DenseNet, MobileNet, and EfficientNet.

In some embodiments of the method, the soil lightness relates to a lightness value in CIELAB color space.

In some embodiments of the method, the soil lightness is normalized based on a parameter selected from the group consisting of: scene luminance, exposure time, aperture size, ISO, camera make, and camera model.

In some aspects, the invention relates to a system for determining soil carbon content.

In some embodiments, the system comprises two radiofrequency (RF) nodes, the RF nodes being positioned such that the soil is between the nodes, a camera, a processor communicatively connected to the RF nodes and the camera, and a non-transitory computer readable medium with instructions stored thereon, which when executed by a processor perform steps comprising: acquiring an image of the soil surface via the camera, determining soil lightness of the soil surface based on the image, propagating an RF signal between the two RF nodes, determining the CSI of the soil based on assessing the propagation of the RF signal between the nodes, estimating carbon content of the soil based on the soil lightness and the CSI, the estimation being based on a mathematical model, refining the estimated carbon content, the refinement comprising predicting a first error between the estimated carbon content and the true carbon content using a machine learning model, and adding the first error to the estimated carbon content.

In some embodiments of the method, the steps performed by the processor further comprise estimating the soil water content based on the soil lightness and the CSI, the estimation being based on a mathematical model, refining the estimated soil water content, the refinement comprising predicting a second error between the estimated soil water content and the true soil water content using a machine learning model, and adding the second error to the estimated carbon content.

In some embodiment of the system, the machine learning model is trained on images of soil surfaces, soil CSI, and ground truth carbon content.

In some embodiments of the system, the step of estimating the carbon content of the soil comprises determining the effective permittivity of the soil based on the CSI.

In some embodiments of the system, the RF signal comprises an RF signal selected from the group consisting of: a Wi-Fi signal, an RFID signal, a backscatter signal, a Radar signal, a LoRa signal, an ultra-wide-band signal, and an LTE signal.

In some embodiments of the system, the camera comprises a smartphone camera.

In some embodiments of the system, at least one of the two RF nodes is buried in the soil.

In some embodiments of the system, the machine learning model comprises a CNN.

In some embodiments of the system, the CNN comprises a CNN selected from the group consisting of: ResNet, VGGNet, Inception, DenseNet, MobileNet, and EfficientNet.

In some embodiments, of the system, the soil lightness relates to a lightness value in CIELAB color space.

In some embodiments of the system, the soil lightness is normalized based on a parameter selected from the group consisting of: scene luminance, exposure time, aperture size, ISO, camera make, and camera model.

In some embodiments of the system, the two RF nodes are connected to a WARP board or a Wi-Fi chipset.

In some embodiments of the system, the soil comprises a field soil or a sand compost mixture.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements.

FIG. 1 depicts a schematic of the relationship between soil carbon, soil physical properties, soil color, and radio frequency (RF) wave propagation.

FIG. 2 depicts a schematic of the design overview of Scarf, a novel soil carbon sensing approach. Two RF nodes collect CSI and a smartphone collects soil surface image. The data is sent to a server to determine carbon content with a method combining mathematical computation and machine learning.

FIG. 3 depicts results of a simulation based on a dielectric mixing model for soils with 40% clay, 40% slit and 20% sand. Effective permittivity (unitless) decreases as carbon level increases.

FIG. 4 depicts a graph showing lightness (unitless) of soil surface images. Lightness decreases as volumetric water content (VWC) and carbon content increase.

FIG. 5 depicts results of a simulation based on empirical models of porosity and wilting point for soils containing 100% sand. Both parameters monotonically increase over soil carbon content.

FIG. 6 depicts a graph showing example of soil organic carbon content oc and VWC w computation based on mathematical models. Given a pair of measured permittivity ∈ and soil lightness L, oc and w can be determined from the intersection of the two functions. The estimated oc and w are 3.6 and 26.7, which align with the ground truth oc and w of 3.0 and 23.4.

FIG. 7, comprising FIGS. 7A through 7B, depict antenna deployment for CSI collection. FIG. 7A depicts a schematic of a horizontal antenna deployment for CSI collection. FIG. 7B depicts a schematic of a vertical antenna deployment for CSI collection.

FIG. 8 depicts a schematic of an ML model overview. First, the math model was to get an initial soil carbon estimation, and then the ML model was used to correct the error between the ground truth and the math-computed output.

FIG. 9 depicts an image of long-term experimental farm plots maintained at different carbon contents.

FIG. 10, comprising FIGS. 10A through 10B, depicts exemplary images of two soil types used for experiments. FIG. 10A depicts exemplary images of soils from the field with 1-4% carbon contents. FIG. 10B depicts exemplary images of sand compost mixtures, consisting of sand and compost manure, at 1-4%, 10%, 15%, and 20% carbon contents.

FIG. 11 depicts laboratory results of water retention curves for sand compost mixtures (M1-M4, M10, M15, M20) (solid lines) and field soils (F1-F4) (dashed lines).

FIG. 12, comprising FIGS. 12A through 12D, depicts sand compost mixtures (1-20% carbon) permittivities measured by different devices showing the same trends. Scarf shows more variations than commodity soil sensors. FIG. 12A depicts sand compost mixtures permittivities permittivities measured by Meter TEROS 10. FIG. 12B depicts sand compost mixtures permittivities measured by Decagon GS3. FIG. 12C depicts sand compost mixtures permittivities measured by Scarf. FIG. 12D depicts sand compost mixtures permittivities measured by Scarf vs permittivites measured by Decagon.

FIG. 13, comprising FIGS. 13A though 13D, depicts field soils (1-4% carbon) permittivities measured by different devices. Scarf's results deviate more from the soil sensors than the sand compost mixture case. FIG. 13A depicts field soils permittivities measured by Meter TEROS 10. FIG. 13B depicts field soils permittivities measured by Decagon GS3.

FIG. 13C depicts field soils permittivities measured by Scarf. FIG. 13D depicts field soils permittivities measured by Scarf vs field soils permittivites measured by Decagon.

FIG. 14, comprising FIGS. 14A through 14B, depicts lightness of field soils. FIG. 14A depicts a graph lightness vs Oven VWC (%) for calibrated images. FIG. 14B depicts a graph lightness vs Oven VWC (%) for raw data.

FIG. 15, comprising FIGS. 15A through 15B, depicts sand compost mixtures carbon content and VWC computed by mathematical model. FIG. 15A depicts a graph showing sand compost mixtures carbon content computed by a math model compared to ground truth carbon content. FIG. 15B depicts a graph showing sand compost mixtures VWC computed by a math model compared to ground truth VWC.

FIG. 16, comprising FIGS. 16A through 16B, depicts field soils carbon content and VWC computed by mathematical model. FIG. 16A depicts a graph field soils carbon content computed by a math model compared to ground truth carbon content. FIG. 16B depicts a graph showing field soils VWC computed by a math model compared to ground truth VWC.

FIG. 17 depicts an exemplary computing environment in accordance with some of the embodiments.

FIG. 18 depicts a schematic of an exemplary method for soil sensing.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Method and System for Soil Sensing

In some aspects, the invention relates to a system for soil sensing. In some aspects, the invention relates to a method for soil sensing. Any soil characteristic may be sensed by the method or system. In some embodiments, the sensed soil characteristic comprises soil carbon content and/or soil water content. In some embodiments, the determination of a soil characteristic is based, at least in part, on soil permittivity and/or soil lightness. In some embodiments, the method or system provides continuous monitoring of a soil characteristic. In some embodiments, the method or system provides monitoring over time by sensing a soil characteristic at any frequency. This invention is based, at least in part, on the unexpected discovery that soil characteristics may be effectively determined from soil permittivity and soil lightness by using a combination of mathematical models and machine learning.

The soil may be any soil. In some embodiments, the soil comprises a soil type classified by the United States Department of Agriculture (USDA) (Wikipedia contributors. (2024 November 20). USDA soil taxonomy. Wikipedia. en(dot)wikipedia(dot)org/wiki/USDA_soil_taxonomy). In some embodiments, the soil comprises one or more soils of any order, suborder, great group, subgroup, family, and series as classified by the USDA. In some embodiments, the soil comprises field soil and/or sand compost mixture. The soil may comprise clay, slit, and/or sand in any relative proportion. The soil may comprise any carbon content or water content. The soil may comprise any permittivity, soil texture, porosity, salt content, nutrient content, iron content, potassium content, phosphorus content, nitrogen content, or bulk density.

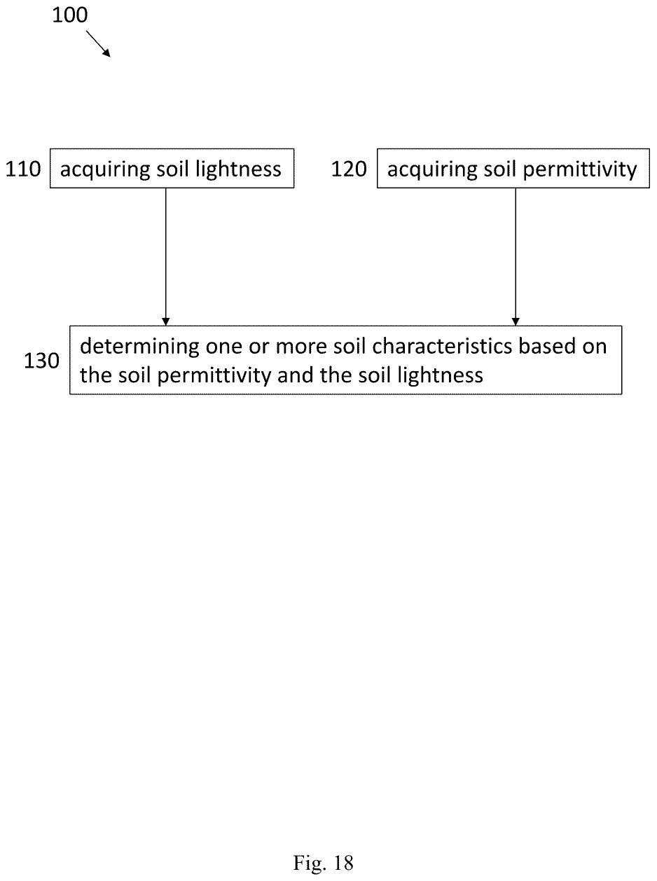

Now referring to FIG. 18, in some embodiments, the method for soil sensing 100 comprises the steps of 110 acquiring soil permittivity, 120 acquiring soil lightness, and/or 130 determining one or more soil characteristics based on the soil permittivity and the soil lightness. In some embodiments, the one or more soil characteristics comprises the soil carbon content and/or the soil water content. In some embodiments, the one or more soil characteristics comprises or further comprises soil content of any nutrient such as soil potassium content and/or soil nitrogen content. In some embodiments, the one or more soil characteristics comprises or further comprises soil texture.

Before soil permittivity and/or soil lightness is acquired, the soil may be manipulated in any fashion to improve the accuracy of the acquired permittivity and soil lightness. For example, the soil may be flattened, raked, and/or any objects may be removed from the soil surface.

In some embodiments, the method 100 comprises the step of 110 acquiring soil permittivity. The soil permittivity may be dielectric permittivity. Dielectric permittivity may be relative permittivity or effective permittivity. Relative permittivity can refer to the ratio of absolute dielectric permittivity to the free-space permittivity. Effective permittivity, also known as apparent permittivity, can refer to the relative permittivity of the soil mixture measured in-situ.

Soil permittivity may be acquired by any suitable method. In some embodiments, soil permittivity is based on a measurement of the propagation of an RF signal in the soil. For example, components of the propagation of an RF signal that may be measured comprise attenuation and/or phase shift. The RF signal may be any RF signal. In some embodiments, the RF signal comprises any RF signal known in the art for detecting soil permittivity. In some embodiments, the RF signal comprises a Wi-Fi signal, an RFID signal, backscatter signal, Radar signal, LoRa signal, ultra-wideband signal, and/or an LTE signal.

In some examples, the propagation of an RF signal in the soil may be measured by capturing channel state information (CSI). Any method for capturing CSI may be used. Exemplary methods for capturing CSI of RF signals in soil are described in Jian Ding and Ranveer Chandra. In The 25th Annual International Conference on Mobile Computing and Networking (MobiCom), pages 1-16. ACM, 2019 which is incorporated herein by reference in its entirety.

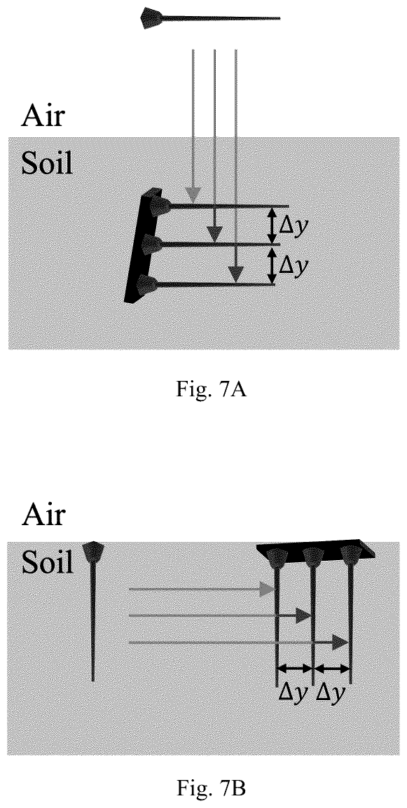

In some embodiments, soil permittivity is determined based on the propagation of a signal in the soil. For example, in some embodiments, two RF nodes are placed at any distance apart such that soil comprises at least part of the distance between the two nodes. One RF node may comprise a transmitter, and one RF node may comprise a receiver. As depicted in FIG. 7A, one RF node may be positioned in the soil and a second RF node may be positioned above the soil. As depicted in FIG. 7B, both RF nodes may be positioned in the soil. An RF node may comprise any number of antennae or any number of antenna arrays. An RF node may be attached to a Wi-Fi chip, a Wi-Fi chipset, a WARP board, an 802.11-complient Wi-Fi Chip, any Wi-Fi device, any Wi-Fi hardware, and/or any commodity Wi-Fi hardware. A smartphone, drone, or any edge device may comprise an RF node for use with the method or system.

Soil permittivity may be calculated or estimated from captured CSI of RF signals in the soil by any method. In some embodiments, relative time-of-flight (ToF) of a signal propagating to two adjacent antennas of an RF node is first determined. Relative ToF (Δt) of an RF signal propagating to two adjacent antennas of an RF node can be simplified to Δt=nΔy/c, where Δy is the distance between antennas, c is the speed of light, and n is the refractive index. n can be related to the effective permittivity ∈ₑ as n=√∈ₑ, therefore Δt=Δy√∈ₑ/c. This relative ToF corresponds to a phase rotation of −2πfΔt, where f is the carrier frequency. In a multipath environment, CSI is the sum of multiple paths, where only the shortest path contains the phase rotation of interest. Pre-processing steps including noise reduction and ToF sanitization may be performed. Exemplary methods for noise reduction and ToF sanitization are described in Manikanta Kotaru, et al. In Proceedings of the 2015 ACM Conference on Special Interest Group on Data Communication (SIGCOMM), pages 269-282, 2015, which is incorporated herein by reference in its entirety. In some embodiments, a multipath resolving technique is used to extract the shortest path and estimate soil effective permittivity. The multipath resolving technique may comprise Multiple Signal Classification (MUSIC). Exemplary methods for extracting the shortest path and/or estimating soil effective permittivity are described in Ralph Schmidt. IEEE transactions on antennas and propagation, 34(3):276-280, 1986 which is incorporated herein by reference in its entirety.

In some embodiments, multiple CSI may be collected for the soil. For example, CSI may be collected for any number of RF transmitter node positions. For example, the RF transmitter node may be moved or rotated any number of times with CSI being collected at every sampled position.

CSI may be collected with the aid of WARP boards, Wi-Fi chipsets, any Wi-Fi hardware, and/or any commodity Wi-Fi hardware. In some embodiments, one or both RF nodes are connected to a WARP board or a Wi-Fi chipset. In some embodiments, the WARP board or Wi-Fi Chipset is not buried in the soil while the connected RF node is buried in the soil. In some embodiments, the WARP board and the connected RF node are both buried in the soil. In some embodiments, CSI is collected at any Wi-Fi frequency. For example, CSI may be collected at or at about 2.4 GHz. In some embodiments, CSI may be collected across any number of channels at a given frequency. In some embodiments, any number of calibration steps may be used when collecting CSI. For example, phase offsets and/or distortions may be removed. Exemplary methods for calibration when collecting CSI are described in Jian Ding and Ranveer Chandra. In The 25th Annual International Conference on Mobile Computing and Networking (MobiCom), pages 1-16. ACM, 2019, which is incorporated herein by reference in its entirety.

In some embodiments, the method 100 comprises the step of 120 acquiring soil lightness. In some embodiments, soil lightness is determined based on an image of the soil. The image of the soil may comprise a soil surface image. Any camera may be used to acquire a soil surface image. In some embodiments, the camera comprises a digital camera or a smartphone camera.

In some embodiments, soil lightness relates to the lightness value of soil of a soil surface image according to CIELAB color space. In some embodiments, a lightness value is assigned to any number of pixels in an image that corresponds to a soil surface. The average, median, or any other descriptive statistic of the lightness value of the pixels or any subset of the pixels in the image that correspond to the soil surface may be used as the soil lightness. For example, the pixel lightness values, corresponding to any region of a soil surface may used to calculate an average or median. The region may be any size.

Soil surface images, including lightness values of pixels of the image, may be calibrated based on any camera setting under which the image was acquired. Camera settings on which image calibration may be based may comprise ISO, exposure time, and/or aperture area. For example, lightness values across images may be normalized based on camera settings based on the following equation: N∝tSLₛ/f², where, N is the pixel lightness value, t is the exposure time, S is the ISO, Lₛ is the scene luminance, and f is the aperture number. Scene luminance may be determined from an image of a reference object under the same or similar imaging conditions. ISO, exposure time, and aperture number may be determined form image metadata. In some embodiments, soil surface images, including pixel lightness values of the image, are calibrated or normalized based on the camera make or camera model of the camera used to acquire the image. In some embodiments, soil surface images, including pixel lightness values of the image, are calibrated or normalized based on any deep-learning based approaches, for example deep-learning based image normalization.

Soil surface images may be cropped, binned, or manipulated using any method to reduce image size.

In some embodiments, the method 100 comprises the step of 130 determining one or more soil characteristics based on the soil permittivity and the soil lightness. In some embodiments, the one or more soil characteristics comprise the soil carbon content and/or the soil water content. In some embodiments, the soil water content comprises soil volumetric water content (VWC). In some embodiments, determining soil carbon content and/or soil water content based on the soil permittivity and the soil lightness comprises the step of estimating soil carbon content and soil water content based on the soil permittivity and soil lightness via a mathematical model, and the step of refining the estimated soil carbon content and estimated soil water content via a machine learning model.

In some embodiments, the mathematical model comprises finding the carbon content, oc, that minimizes the following objective function: f_{obj}=|∈̅ₑ−f_{∈}(L̅, oc)|, where oc is carbon content, ∈̅ₑ and L̅ are the measured effective permittivity and soil lightness, and ∈ₑ=f_{∈} is the mathematical model of effective permittivity, which is a function of L̅ and oc. f_{∈}(L̅, oc) is derived from f_{∈}(w, oc), where w is soil VWC. w can be written as a function of L̅ and oc as w=[−a|n(oc+1)+b−L̅]/c, where a, b, c>0 are constants. f_{∈}(w, oc) is rewritten from f_{∈}(w, w_{wp}, p), since VWC of the soil at wilting point, w_{wp}, and porosity of the soil, p, are each dependent on soil carbon content, oc, for a given soil type. f_{∈}(w, w_{wp}, p) is derived from

$${ϵ}_{e} = \{ \begin{matrix}( 1 - p ) {ϵ}_{s} + ( p - w ) {ϵ}_{a} + w {ϵ}_{b} & w \leq {w}_{wp} \\ ( 1 - p ) {ϵ}_{s} + ( p - w ) {ϵ}_{a} + w ( \frac{p - w}{p - {w}_{wp}} {ϵ}_{b} + \frac{w - {w}_{wp}}{p - {w}_{wp}} {ϵ}_{f} ) & {w}_{wp} < w \leq p \\ ( 1 - w ) {ϵ}_{s} + w {ϵ}_{f} & w > p\end{matrix}$$

where ∈ₛ, ∈ₐ, ∈_{b} and ∈_{f} are the permittivity of soil, air, bound water and free water, respectively. In other words, the mathematical model may comprise finding the carbon content, oc, for which the value of fₑ(L̅, oc) is closest to the acquired soil permittivity. Relevant equations, including equations for soil porosity, p, are described in Chang-Hwan Park et al. which is incorporated herein by reference in its entirety (Chang-Hwan Park, et al., Vadose Zone Journal, 18(1):190036, 201).

In some embodiments, the VWC of the soil at wilting point, w_{wp}, is measured. In some embodiments, any value known in the art for the VWC of the soil at wilting point, w_{wp}, is used. In some examples, w_{wp}, is dependent on the carbon content of the soil, the clay content of the soil, the type of clay in the soil, the sand content of the soil, organic matter content of the soil, soil structure, soil salinity, and/or soil bulk density. Soils with more clay may have a higher w_{wp}. Soils with more sand may have a lower w_{wp}. Soils comprising smectite clays may have a higher w_{wp}. Soils with more kaolinite clay may have a lower w_{wp}. Soils with more organic matter may have a higher w_{wp}. Soils with higher salinity may have a higher w_{wp}. In some embodiments, w_{wp}, is computed from the following equation w_{wp}=0.0298+0.089v_{clay}+0.01360c, where v_{clay} is the volumetric ratio of clay in soil and oc is the organic carbon content (%). In some embodiments, w_{wp}, is in the range of 5% to 12%. Exemplary methods for determining VWC of the soil at wilting point, soil porosity, and soil bulk density are described in Chang-Hwan Park, et al., Vadose Zone Journal, 18(1):190036, 2019 and Manikanta Kotaru, et al., In Proceedings of the 2015 ACM Conference on Special Interest Group on Data Communication (SIGCOMM), pages 269-282, 2015 which are each incorporated herein by reference in their entirety.

In some embodiments, soil porosity, p, is modeled as a non-linear function of soil carbon content, oc, soil bulk density, BD, and volumetric ratios of clay, v_{clay}, and slit, vₛₗᵢₜ, in soil.

In some embodiments, the estimated carbon content, denoted as o̅c̅, derived from the mathematical model is used to determine an estimated VWC according to the following equation: w̅= [−a|n(oc+1)+b−L̅]/c, where w̅ is the estimated VWC.

Constants, a, b, and, c may be determined empirically from measured soil carbon content, soil VWC and soil lightness values. Given a set of measured carbon content, oc, VWC, w, and lightness, L, values, i.e., oc₁, oc₂, . . . , ocₙ, w₁, w₂, . . . , wₙ, and L₁, L₂, . . . , Lₙ, the constants, a, b and c may be computed by solving the following equation:

$${\underbrace{[ \begin{matrix}- ln ( {oc}_{1} + 1 ) & 1 & - {w}_{1} \\ - ln ( {oc}_{2} + 1 ) & 1 & - {w}_{2} \\ ⋮ & ⋮ & ⋮ \\ - ln ( {oc}_{n} + 1 ) & 1 & - {w}_{n}\end{matrix} ]}}_{A} {\underbrace{[ \begin{matrix}a \\ b \\ c\end{matrix} ]}}_{x} = {\underbrace{[ \begin{matrix}{L}_{1} \\ {L}_{2} \\ ⋮ \\ {L}_{N}\end{matrix} ]}}_{B}$$

In some embodiments, the constant, a, may be 30 or about 30. In some embodiments, the constant, b, may be 100 or about 100. In some embodiments, the constant, c, may be in the range of 0.5 to 3 or about 0.5 to about 3.

The permittivity of soil, ∈ₛ, may depend partly or may depend solely on soil texture and soil type. Therefore, ∈ₛ, may be a constant for a soil with a given soil texture and soil type. In some embodiments, ∈ₛ is computed from the volumetric ratios of sand, silt and clay in soil and their respective permittivities. For example, the volumetric proportion of sand, v_{sand}, clay, v_{clay}, and silt, vₛᵢₗₜ, as well as the permittivity of sand ∈_{sand}, silt, ∈ₛᵢₗₜ, and clay, ∈_{clay}, may be used to compute ∈ₛ according to the following formula: ∈ₛ=v_{sand}∈_{sand}+vₛᵢₗₜ ∈ₛᵢₗₜ+v_{clay}∈_{clay}. In some embodiments, the permittivity of sand, silt, and soil are measured. In some embodiments, any values known in the art for the permittivity of sand, silt, and soil are used. In some embodiments, the permittivity of sand is 3+0.078j or is about 3+0.078j. In some embodiments, the permittivity of silt is 5+0.078j or is about 5+0.078j. In some embodiments, the permittivity of clay is 5+0.078j or is about 5+0.078j. Exemplary values for the permittivities of sand, soil and clay are described in Chang-Hwan Park et al. which is incorporated herein by reference in its entirety (Chang-Hwan Park, et al., Remote Sensing, 9(7):732, 2017).

In some embodiments, the permittivity of air, ∈ₐ, is measured. In some embodiments, any value known in the art for the permittivity of air, ∈ₐ, is used. In some embodiments, the value of ∈ₐ is 1 or about 1.

The permittivity of bound water, ∈_{b}, and the permittivity of free water, ∈_{f}, may depend on operating frequency and/or soil temperature. In some embodiments, the permittivity of bound water, ∈_{b}, and the permittivity of free water, ∈ᵣ, are measured. In some embodiments, any value known in the art for the permittivity of bound water, ∈_{b}, and any value known in the art for the permittivity of free water, ∈ᵣ, are used. Exemplary methods for determining the permittivity of bound water, ∈_{b}, and the permittivity of free water, ∈ᵣ, are described in Chang-Hwan Park, et al., Vadose Zone Journal, 18(1):190036, 2019 which is incorporated herein by reference in its entirety. The permittivity of bound water may be in the range of the permittivity of ice to the permittivity of free water at 20 °C. The permittivity of ice may be 3.2 or may be about 3.2. The permittivity of free water at 20 °C may be 80 or may be about 80.

In some embodiments, the estimated soil VWC and estimated soil carbon content determined from the mathematical model are refined via a machine learning model. In some embodiments, the machine learning model is trained on the soil surface image that was used to acquire soil lightness of a soil, CSI that was used to acquire soil permittivity of the soil, and the difference between the estimated soil carbon content via the mathematical model and the ground truth soil carbon content.

The machine learning model may be any model comprising any model architecture. In some embodiments, the machine learning model is applied using cloud-based processing. In some embodiments, the machine learning model comprises a convolutional neural network (CNN). The CNN may be any suitable CNN. The CNN may comprise any modifications including any depth, width, convolution kernel size, activation functions, normalization techniques, or other hyperparameters. In some embodiments, the CNN comprises ResNet VGGNet, Inception, DenseNet, MobileNet, EfficientNet, and/or any combinations variants thereof (Kaiming He, et al., In Proceedings of the IEEE conference on computer vision and pattern recognition (CVPR), pages 770-778, 2016.). In some embodiments, ResNet comprises a ResNet regression model. In some embodiments, the ResNet regression model is implanted with a deep learning library, for example Pytorch (Adam Paszke, et al., Advances in neural information processing systems, 32, 2019). In some embodiments, a Stochastic Gradient Descent (SGD) optimizer is used with the CNN. Any batch size and any number of layers may be used with the CNN. In some embodiments, a batch size of 16 is used. In some embodiments, 14 layers are used. The CNN may be run on any GPU, for example NVIDIA GeForce RTX 4070 GPU.

The CSI input to the machine learning model may comprise any format that retains phase and amplitude information. The CSI input to the machine learning model may comprise any size matrix of complex numbers. The matrix may be converted to an image before input to the machine learning model. The matrix size may be 3×330. In some embodiments, the CSI input to the machine learning model may comprise CSI represented on the MUSCI spectrum. For example, CSI input represented on the MUSIC spectrum, may be presented as a relative ToF-absolute ToF heatmap. Exemplary signal processing algorithms that may be used with CSI before inputting to the machine learning model are described in Jian Ding and Ranveer Chandra. In The 25th Annual International Conference on Mobile Computing and Networking (MobiCom), pages 1-16. ACM, 2019 and Manikanta Kotaru, et al., In Proceedings of the 2015 ACM Conference on Special Interest Group on Data Communication (SIGCOMM), pages 269-282, 2015 which are each incorporated herein by reference in their entirety.

The soil surface images inputted to the machine learning model may comprise smartphone images. The images may be any size. The images may comprise any number of pixels. In some embodiments, the images are cropped or binned before being inputted. In some embodiments, the images are cropped and/or binned such that the images are an image size that is commonly used for any CNN.

Ground truth soil carbon content for the purposes of training a machine learning model may be acquired or measured by any method. In some embodiments, soil carbon content is measured by a standard dry combustion technique. In some embodiments, soil samples may be taken and carbon content of the samples may be measured using a Flash 2000 NC soil analyzer. Exemplary methods for measuring soil carbon content are described in Darrell W Nelson and Lee E Sommers. Methods of soil analysis: Part 3 Chemical methods, 5:961-1010, 1996 which is hereby incorporated by reference in its entirety.

In some embodiments, the invention provides a system for determining soil carbon and/or soil water content. In some embodiments, the system comprises a computing device, an imaging device, and/or RF nodes. In some embodiments, the imaging device and RF nodes are communicatively connected to the computing device.

The RF nodes of the system may comprise any RF nodes described herein. For example, the system may comprise two RF nodes placed at any distance apart such that soil designated for sensing comprises at least part of the distance between the two nodes. For example, in some embodiments, one RF node of the system comprises a transmitter, and one RF node of the system comprises a receiver. As depicted in FIG. 7A, one RF node may be positioned in the soil and a second RF node may be positioned above the soil. As depicted in FIG. 7B, both RF nodes may be positioned in the soil. An RF node may comprise any number of antennae or any number of antenna arrays. An RF node may be attached to a Wi-Fi chip, a Wi-Fi chipset, a WARP board, an 802.11-complient Wi-Fi Chip, any Wi-Fi device, any Wi-Fi hardware, and/or any commodity Wi-Fi hardware. A smartphone, drone, or any edge device may comprise an RF node for use with the system.

The imaging device of the system may comprise any suitable image device for imaging soil. The imaging device may be positioned appropriately such that it may image the soil designated for sensing. The imaging device may comprise any camera suitable for acquiring any images described herein. The imaging device may comprise any digital camera or any smartphone camera.

The computing device of the system may comprise any computing device suitable for performing any calculations described herein.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

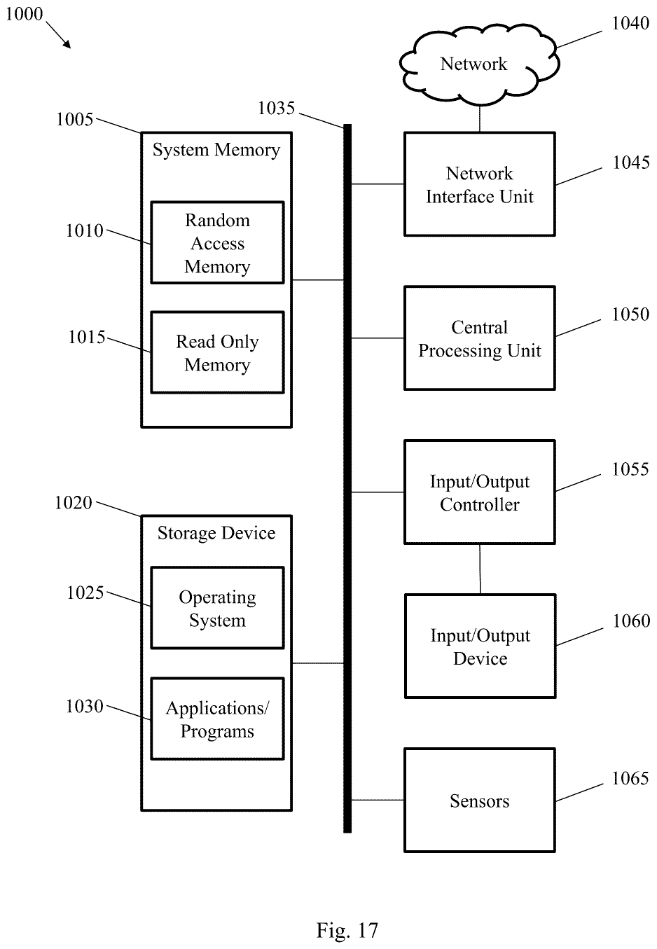

FIG. 17 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 17 depicts an illustrative computer architecture for a computer 1000 for practicing the various embodiments of the invention. The computer architecture shown in FIG. 17 illustrates a conventional personal computer, including a central processing unit 1050 (“CPU”), a system memory 1005, including a random access memory 1010 (“RAM”) and a read-only memory (“ROM”) 1015, and a system bus 1035 that couples the system memory 1005 to the CPU 1050. A basic input/output system containing the basic routines that help to transfer information between elements within the computer, such as during startup, is stored in the ROM 1015. The computer 1000 further includes a storage device 1020 for storing an operating system 1025, application/program 1030, and data.

The storage device 1020 is connected to the CPU 1050 through a storage controller (not shown) connected to the bus 1035. The storage device 1020 and its associated computer-readable media provide non-volatile storage for the computer 1000. Although the description of computer-readable media contained herein refers to a storage device, such as a hard disk or CD-ROM drive, it should be appreciated by those skilled in the art that computer-readable media can be any available media that can be accessed by the computer 1000.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1000 may operate in a networked environment using logical connections to remote computers through a network 1040, such as TCP/IP network such as the Internet or an intranet. The computer 1000 may connect to the network 1040 through a network interface unit 1045 connected to the bus 1035. It should be appreciated that the network interface unit 1045 may also be utilized to connect to other types of networks and remote computer systems.

The computer 1000 may also include an input/output controller 1055 for receiving and processing input from a number of input/output devices 1060, including a keyboard, a mouse, a touchscreen, a camera, a microphone, a controller, a joystick, or other type of input device. Similarly, the input/output controller 1055 may provide output to a display screen, a printer, a speaker, or other type of output device. The computer 1000 can connect to the input/output device 1060 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 1020 and/or RAM 1010 of the computer 1000, including an operating system 1025 suitable for controlling the operation of a networked computer. The storage device 1020 and RAM 1010 may also store one or more applications/programs 1030. In particular, the storage device 1020 and RAM 1010 may store an application/program 1030 for providing a variety of functionalities to a user. For instance, the application/program 1030 may comprise many types of programs such as a word processing application, a spreadsheet application, a desktop publishing application, a database application, a gaming application, internet browsing application, electronic mail application, messaging application, and the like. According to an embodiment of the present invention, the application/program 1030 comprises a multiple functionality software application for providing word processing functionality, slide presentation functionality, spreadsheet functionality, database functionality and the like.

The computer 1000 in some embodiments can include a variety of sensors 1065 for monitoring the environment surrounding and the environment internal to the computer 1000. These sensors 1065 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Example #1—Cost-Effective Soil Carbon Sensing with Wi-Fi and Optical Signals

In this Example, Scarf, a novel soil carbon sensing approach that combines widely accessible radio frequency (RF) and optical signals to detect soil carbon contents without dedicated hardware, is described. The key insight is that soil carbon content closely correlates with two indicators: the effective permittivity derived from RF signals and soil lightness determined from soil surface images. The correlations were mathematically modeled by leveraging the non-linear correlation between the two signal modalities to compute soil carbon content. Machine learning was employed to model relationships that cannot be captured by traditional mathematical equations. The experimental results indicated that Scarf achieves high soil carbon prediction accuracy that is comparable to the state-of-the-art soil carbon sensing techniques which cost US$1000s.

Over the past few decades, soil carbon has emerged as a critical focus for researchers and farmers, driven by its pivotal role in climate change, soil health, and agricultural sustainability. As part of the global carbon cycle (GCC), soil carbon helps remove carbon dioxide (CO2) from the atmosphere through a process known as carbon sequestration. During this process, CO2 is first absorbed by plants through photosynthesis, followed by the transfer of a portion of the carbon into soil through the decomposition of plant material. Soil carbon is also crucial in fostering soil health since it is closely correlated with the retention and use efficiency of water and nutrients in soil (Rattan Lal. Food and Energy Security, 5(4):212-222, 2016.). Soil with higher carbon content can hold more water and nutrients, thus leading to higher crop productivity. Furthermore, the knowledge of soil carbon empowers farmers to adopt sustainable agriculture practices that optimize resource efficiency, including water and fertilizer use, while simultaneously enhancing crop productivity and environmental protection (Rattan Lal. Soils and Agronomy for Sustainable Development, 28:57-64, 2008.).

Traditional methods for measuring soil carbon are expensive and time-consuming. The standard method is dry combustion, which requires longer than one week of time and can only measure a small volume of soil (Amitava Chatterjee, et al., Critical Reviews in Plant Science, 28(3):164-178, 2009., Darrell W Nelson and Lee E Sommers. Methods of soil analysis: Part 3 Chemical methods, 5:961-1010, 1996.). Moreover, it costs around US$20 per soil sample and therefore is not suitable for monitoring small soil carbon changes over large areas or over time (The Service Testing and Research Laboratory at Ohio State University. Sample analysis fee schedule. https://bpb-us-w2(dot)wpmucdn.com/u(dot)osu(dot)edu/dist/4/39220/files/2022/07/STAR-lab-analysis-cost_FY-23(dot)pdf, September 2022., SWPT Laboratory at Colorado State University. Feed and forage analysis price list. https://agsci.colostate(dot)edu/soiltestinglab/price-list/,May 2023.). There are also in-situ methods such as laser-induced spectroscopy and inelastic neutron scattering (David A Cremers, et al., Journal of Environmental Quality, 30(6):2202-2206, 2001., Michael H Ebinger, et al., Carbon sequestration in soils of Latin America. Food Products Press, New York, pages 407-422, 2006., Lucian Wielopolski, et al., Soil Science Society of America Journal, 72(5):1269-1277, 2008.). However, these methods typically cost more than US$10k (Amitava Chatterjee, et al., Critical Reviews in Plant Science, 28(3):164-178, 2009). There have been plenty of efforts in developing lower-cost soil carbon sensing systems utilizing soil color and reflectance (Skye A Wills, et al., Soil Science Society of America Journal, 71(2):380-388, 2007., Naoki Moritsuka, et al., Soil science and plant nutrition, 60(4):475-485, 2014., R A Viscarra Rossel, et al., Biosystems engineering, 100(2):149-159, 2008., Luis Gómez-Robledo, et al., Computers and electronics in agriculture, 99:200-208, 2013., Yijia Tang, et al., Geoderma Regional, 20:e00240, 2020., Patrick M Ewing, et al., Soil Science Society of America Journal, 85(5):1814-1826, 2021., Amin Sharififar, et al., Soil Use and Management, 35(4):607-616, 2019.). However, the trade-off for reduced cost is often compromised accuracy in these systems. Additionally, these systems often require soil sample preparation through air drying and sieving, which hinders their ability to obtain rapid carbon measurements. No existing system can fulfill the combined requirements of accurate, inexpensive and rapid determination of soil carbon. According to the soil scientists that were consulted, there is a significant demand for such systems.

In this Example, a system that meets all the requirements mentioned above was developed. Signals in RF and optical bands, i.e., Wi-Fi and images, which are widely available on edge devices like smartphones and drones, were utilized for soil carbon sensing. Recent research has demonstrated that various RF signals, including Wi-Fi, RFID, LTE, etc., can be used to detect soil permittivity (Jian Ding and Ranveer Chandra. In The 25th Annual International Conference on Mobile Computing and Networking (MobiCom), pages 1-16. ACM, 2019., Colleen Josephson, et al., In ACM SIGCAS Conference on Computing and Sustainable Societies, pages 299-311, 2021., Zhaoxin Chang, et al., Proceedings of the ACM on Interactive, Mobile, Wearable and Ubiquitous Technologies, 6(2):1-27, 2022., Yuda Feng, et al., In Proceedings of the 20th ACM Conference on Embedded Networked Sensor Systems (SenSys), pages 421-434, 2022.). In addition, soil studies have shown that soil carbon content influences soil permittivity (Valery Mironov and Igor Savin. Physics and Chemistry of the Earth, Parts A/B/C, 83:57-64, 2015., Chang-Hwan Park, et al., Vadose Zone Journal, 18(1):190036, 2019.). Collectively, these insights suggested a potential correlation between RF signals and soil carbon, with soil permittivity as an intermediate variable. However, a challenge here is that soil permittivity changes caused by soil carbon and soil moisture are entangled. That means, only measuring soil permittivity is inadequate to determine soil carbon content, necessitating additional information. To complement the RF data, images, which are easily accessible on smartphones and have been shown to correlate with soil carbon content through color analysis, were utilized (Skye A Wills, et al., Soil Science Society of America Journal, 71(2):380-388, 2007., Naoki Moritsuka, et al., Soil science and plant nutrition, 60(4):475-485, 2014., Albert Henry Munsell. A Color Notation: A Measured Color System Based on the Three Qualities, Hue, Values and Chroma with Illustrative Models, Charts and a Course of Study Arranged for Teachers. Geo. H. Ellis, 1907.). The traditional color-based carbon sensing methods, however, rely on known soil moisture levels. They require separately measuring soil moisture or air drying the soil sample. Motivated by the aforementioned insights, Scarf, a system for Soil carbon sensing using RF signals and images, was designed and implemented.

### Major Contributions of Scarf

Scarf, described herein, is a novel soil carbon sensing technique that enables low-cost, rapid and flexible soil carbon detection with RF signals and images. Scarf is the first work to enable soil carbon sensing with commodity wireless devices, whereas existing low-cost RF-based soil sensing techniques only focus on soil moisture (as shown in Table 1, below). As a proof-of-concept, Scarf was implemented with commodity WARP hardware and smartphone camera without modification, demonstrating the possibility to bring down the cost of soil carbon sensing to US$10s (Rice University. Warp project. http://warpproject(dot)org.).

TABLE 1
Soil properties measured by recent RF-based sensing
techniques. Scarf is the only RF-based sensing
technique that measures soil carbon.

| Method | Soil Property |
|---|---|
| GreenTag (RFID) (Ju Wang, et al., In | Moisture |
| Proceedings of the 18th International | |
| Conference on Mobile Systems, Applications, | |
| and Services (MobiSys), pages 273-285, 2020) | |
| Backsatter (Colleen Josephson, et al., In ACM | Moisture |
| SIGCAS Conference on Computing and | |
| Sustainable Societies, pages 299-311, 2021) | |
| Smol (LoRa) (Daniel Kiv, et al.,. In | Moisture |
| Proceedings of the 1st ACM Workshop on No | |
| Power and Low Power Internet-of-Things, | |
| pages 21-27, 2022.) | |
| LoRa (Zhaoxin Chang, et al., Proceedings of | Moisture |
| the ACM on Interactive, Mobile, Wearable and | |
| Ubiquitous Technologies, 6(2): 1-27, 2022.) | |
| CoMEt (USRP) (Usman Mahmood Khan and | Moisture |
| Muhammad Shahzad. In Proceedings of the | |
| 28th Annual International Conference on | |
| Mobile Computing and Networking | |
| (MobiCom), pages 242-254, 2022.) | |
| LTE-Soil-Meter (Yuda Feng, et al., In | Moisture |
| Proceedings of the 20th ACM Conference on | |
| Embedded Networked Sensor Systems | |
| (SenSys), pages 421-434, 2022.) | |
| Strobe (Wi-Fi) (Jian Ding and Ranveer | Moisture and EC |
| Chandra. In The 25th Annual International | |
| Conference on Mobile Computing and | |
| Networking (MobiCom), pages 1-16. ACM, | |
| 2019.) | |
| Scarf (This Example) | Carbon |

Mathematical models were derived to show that estimating soil carbon from RF signals and images is theoretically feasible because the impacts of soil carbon and soil moisture on RF signals and images are non-linearly correlated. This Example is the first work to demonstrate the possibility of using a combination of RF signals and images to sense soil carbon.

Given the limitations of mathematical models in fully capturing the complex relationships among soil carbon, moisture, permittivity, and lightness, a machine learning component was incorporated. The resulting model can better handle the complexities inherent in soil systems by combining the strengths of both mathematical modeling and machine learning.

Scarf was tested with two soil types. The evaluation results showed that Scarf's approach of combining mathematical computation with machine learning can help improve the accuracy of mathematical models, even with small-size datasets. For sand compost mixtures, Scarf achieves a high correlation of 96.3% and coefficient of determination (R²) of 0.911, which is comparable to the state-of-the-art soil carbon sensing techniques using devices that cost US$1000s (Table 2, below) (Yijia Tang, et al., Geoderma Regional, 20:e00240, 2020., Amin Sharififar, et al., Soil Use and Management, 35(4):607-616, 2019.). For field soils, Scarf achieved slightly worse performance with a correlation of 88.0% and R² of 0.417, due to the relatively small dataset and the small range of carbon content.

TABLE 2
Low-cost soil carbon sensing techniques. Scarf achieved
high accuracy without the need for sample drying.

| | | Sample | |
|---|---|---|---|
| Method | Accuracy (R²) | drying | Price (USD) |
| Chroma meters (Skye A | 0.53-0.79 | Yes | 1000 s |
| Wills, et al., Soil | | | |
| Science Society of | | | |
| America Journal, | | | |
| 71(2): 380-388, 2007., | | | |
| Naoki Moritsuka, et al., | | | |
| Soil science and plant | | | |
| nutrition, 60(4): 475- | | | |
| 485, 2014.) | | | |
| Digital Camera (RA | 0.88 | Yes | 100 s-1000 s |
| Viscarra Rossel, et al., | | | |
| Biosystems engineering, | | | |
| 100(2): 149-159, 2008.) | | | |
| Remote Sensing | 0.23-0.89 | No | depends |
| (Theodora | | | |
| Angelopoulou, et al., | | | |
| Remote Sensing, | | | |
| 11(6): 676, 2019., Umesh | | | |
| Acharya, et al., Carbon | | | |
| Management, 13(1): 401- | | | |
| 419, 2022.) | | | |
| Spectrometers (Yijia | 0.73-0.78 | Yes | 1000 s |
| Tang, et al., Geoderma | | | |
| Regional, 20: e00240, | | | |
| 2020., Amin Sharififar, | | | |
| et al., Soil Use and | | | |
| Management, | | | |
| 35(4): 607-616, 2019.) | | | |
| Reflectometers (Patrick | 0.57 | Yes | 350 |
| M Ewing, et al., Soil | | | |
| Science Society of | | | |
| America Journal, | | | |
| 85(5): 1814-1826, | | | |
| 2021.) | | | |
| Scarf (this Example) | 0.91 | No | 10 s |

### Related Work

### Low-Cost Soil Carbon Sensing.

The efforts on low-cost soil carbon sensing can be divided into two categories. (i) Soil color-based methods leverage the relationship between soil color and soil carbon. Soil color can be determined visually with Munsell color charts or more accurately with chroma meters (Skye A Wills, et al., Soil Science Society of America Journal, 71(2):380-388, 2007., Naoki Moritsuka, et al., Soil science and plant nutrition, 60(4):475-485, 2014., Albert Henry Munsell. A Color Notation: A Measured Color System Based on the Three Qualities, Hue, Values and Chroma with Illustrative Models, Charts and a Course of Study Arranged for Teachers. Geo. H. Ellis, 1907.). Recent studies demonstrated the possibility of using digital cameras and cameras on mobile phones to measure soil color (RA Viscarra Rossel, et al., Biosystems engineering, 100(2):149-159, 2008., Luis Gómez-Robledo, et al., Computers and electronics in agriculture, 99:200-208, 2013.). (ii) Reflectance-based methods rely on the relationship between reflectance spectra and soil organic carbon in the visible, near-infrared and shortwave infrared (400-2500 nm) regions (Theodora Angelopoulou, et al., Remote Sensing, 11(6):676, 2019., Sidney Allen Bowers. Kansas State University, 1971., Eyal Ben-Dor, et al., Remote sensing of Environment, 61(1):1-15, 1997.). To reduce the cost of soil carbon sensing, spectrometers can be deployed on satellites, aircraft, and unmanned aerial systems to provide large-scale and rapid soil carbon sensing. These remote sensing systems, however, achieve lower accuracy and suffer from weather conditions and soil conditions such as vegetation cover, soil moisture and soil roughness (Theodora Angelopoulou, et al., Remote Sensing, 11(6):676, 2019., Marco Nocita, et al., Geoderma, 199:37-42, 2013.). Recent studies also look into portable devices using low-cost spectrometers, which have limited spectral ranges, for soil carbon determination. Compared to more capable instruments, these simplified devices can bring down the cost from ~US$50,000 to US$100s-1000s (Yijia Tang, et al., Geoderma Regional, 20:e00240, 2020., Patrick M Ewing, et al., Soil Science Society of America Journal, 85(5):1814-1826, 2021., Amin Sharififar, et al., Soil Use and Management, 35(4):607-616, 2019.). A field portable reflectometer, which costs US$350, has been demonstrated but only achieves an accuracy of 0.57 (Patrick M Ewing, et al., Soil Science Society of America Journal, 85(5):1814-1826, 2021).

As shown in Table 2, for all these approaches, except remote sensing, a key limitation is that soil samples require drying and sieving before conducting measurements, leading to extra time and labor effort (Table 2, above). Although remote sensing does not need sample preparation, its performance is not stable. Scarf adopts two signal modalities to eliminate the need for sample drying while achieving good accuracy.

### Low-Cost RF-Based Soil Sensing

Recently, several novel sensing techniques have been proposed to enable low-cost and accurate soil moisture sensing using RF signals. In contrast to traditional RF-based sensing techniques such as ground penetrating radar (GPR) and Time Domain Reflectometry (TDR), which requires expensive specialized hardware, the recent studies focus on low-cost commodity wireless devices that bring down the cost from US$1000s to under US$100 (Johan Alexander Huisman, et al., Vadose zone journal, 2(4):476-491, 2003., Kosuke Noborio. Computers and electronics in agriculture, 31(3):213-237, 2001.). Strobe proposes using Wi-Fi signals for soil moisture and electrical conductivity (EC) sensing (Jian Ding and Ranveer Chandra. In The 25th Annual International Conference on Mobile Computing and Networking (MobiCom), pages 1-16. ACM, 2019.). The authors in Josephenson et al. use low-cost and low-energy backscatter to sense soil moisture leveraging ultra-wideband signals (Colleen Josephson, et al., In ACM SIGCAS Conference on Computing and Sustainable Societies, pages 299-311, 2021.). GreenTag leverages Differential Minimum Response Threshold (DMRT) of two commodity RFID tags to detect soil moisture of plant pots in a greenhouse (Ju Wang, et al., In Proceedings of the 18th International Conference on Mobile Systems, Applications, and Services (MobiSys), pages 273-285, 2020.). Recent work also uses LoRa signals for soil moisture sensing (Zhaoxin Chang, et al., Proceedings of the ACM on Interactive, Mobile, Wearable and Ubiquitous Technologies, 6(2):1-27, 2022., Daniel Kiv, et al., In Proceedings of the 1st ACM Workshop on No Power and Low Power Internet-of-Things, pages 21-27, 2022.). The authors in Chang et al. use the relative time-of-flight (ToF) between two receive antennas to sense soil moisture (Zhaoxin Chang, et al., Proceedings of the ACM on Interactive, Mobile, Wearable and Ubiquitous Technologies, 6(2):1-27, 2022.). Smol only adopts received signal strength indicator (RSSI) information, which limits the estimation accuracy (Daniel Kiv, et al., In Proceedings of the 1st ACM Workshop on No Power and Low Power Internet-of-Things, pages 21-27, 2022.). COMEt is a non-invasive system with both the transmitter and receive antenna array above the soil surface (Usman Mahmood Khan and Muhammad Shahzad. In Proceedings of the 28th Annual International Conference on Mobile Computing and Networking (MobiCom), pages 242-254, 2022.). LTE-Soil-Meter measures the relative ToF of two LTE receivers (Yuda Feng, et al., In Proceedings of the 20th ACM Conference on Embedded Networked Sensor Systems (SenSys), pages 421-434, 2022.).

As shown in Table 1, most existing techniques only sense soil moisture (Table 1, above). Scarf is the only work to sense soil carbon. As a proof-of-concept, this work chooses Wi-Fi signals to detect soil permittivity. However, Scarf's mathematical models may also applicable to other RF signals which can detect soil permittivity, e.g., LTE signals, after adapting the operating frequency in the equations (Colleen Josephson, et al., In ACM SIGCAS Conference on Computing and Sustainable Societies, pages 299-311, 2021., Zhaoxin Chang, et al., Proceedings of the ACM on Interactive, Mobile, Wearable and Ubiquitous Technologies, 6(2):1-27, 2022., Yuda Feng, et al., In Proceedings of the 20th ACM Conference on Embedded Networked Sensor Systems (SenSys), pages 421-434, 2022., Usman Mahmood Khan and Muhammad Shahzad. In Proceedings of the 28th Annual International Conference on Mobile Computing and Networking (MobiCom), pages 242-254, 2022.).

### Application of Machine Learning in RF Sensing

In recent years, there have been rich applications of machine learning techniques in RF sensing for human activities, such as gesture recognition, fall detection, pose estimation, etc. (Wenjun Jiang, et al., In Proceedings of the 24th annual international conference on mobile computing and networking (MobiCom), pages 289-304, 2018., Yonglong Tian, et al., Proceedings of the ACM on Interactive, Mobile, Wearable and Ubiquitous Technologies, 2(3):1-24, 2018., Mingmin Zhao, et al., In Proceedings of the 2018 Conference of the ACM Special Interest Group on Data Communication (SIGCOMM), pages 267-281, 2018., Mingmin Zhao, et al., In Proceedings of the IEEEConference on Computer Vision and Pattern Recognition (CVPR), pages 7356-7365, 2018., Shuya Ding, et al., In Proceedings of the 18th Conference on Embedded Networked Sensor Systems, pages 517-530, 2020., Tianhong Li, et al., In Proceedings of the IEEE/CVF Winter Conference on Applications of Computer Vision, pages 3288-3297, 2022., Ruiyuan Song, et al., In Proceedings of the 28th Annual International Conference on Mobile Computing And Networking (Mobi-Com), pages 282-295, 2022.) These systems typically adopt convolutional neural network (CNN), more specifically, ResNet, to extract features from RF data (GW McCarty, et al., Soil Science Society of America Journal, 66(2):640-646, 2002.). This is because CNN has demonstrated superior performance in human activity detection in images and videos while ResNet has its advantages of allowing deeper neural architectures, making it possible to detect more complex patterns. Earlier systems mostly rely on supervised learning architectures, which requires ground truth labels provided by other signal modalities such as RGB data (Yonglong Tian, et al., Proceedings of the ACM on Interactive, Mobile, Wearable and Ubiquitous Technologies, 2(3):1-24, 2018., Mingmin Zhao, et al., In Proceedings of the 2018 Conference of the ACM Special Interest Group on Data Communication (SIGCOMM), pages 267-281, 2018., Mingmin Zhao, et al., In Proceedings of the IEEE Conference on Computer Vision and Pattern Recognition (CVPR), pages 7356-7365, 2018.). To reduce the laborious labeling efforts, several systems are developed to utilize partially labeled datasets [35, 39] and unsupervised learning architectures (Wenjun Jiang, et al., In Proceedings of the 24th annual international conference on mobile computing and networking (MobiCom), pages 289-304, 2018., Shuya Ding, et al., In Proceedings of the 18th Conference on Embedded Net-worked Sensor Systems, pages 517-530, 2020., Tianhong Li, et al., In Proceedings of the IEEE/CVF Winter Conference on Applications of Computer Vision, pages 3288-3297, 2022., Ruiyuan Song, et al., In Proceedings of the 28th Annual International Conference on Mobile Computing And Networking (Mobi-Com), pages 282-295, 2022.). Scarf differs from these systems in its utilization of RGB data, which is combined with RF signals as the input to the machine learning model.

### Soil Carbon Primer

Soil carbon includes both inorganic and organic carbon. Inorganic carbon exists in mineral forms, primarily as calcium carbonate, while organic carbon presents as soil organic matter—a complex mixture of decomposed plant and animal residues. In this Example, soil organic carbon was focused on, given its greater responsiveness to agriculture practices (Amitava Chatterjee, et al., Critical Reviews in Plant Science, 28(3):164-178, 2009.). Throughout the Example, soil carbon is used to refer to the organic portion of soil carbon.

The standard method of measuring soil carbon is dry combustion (Amitava Chatterjee, et al., Critical Reviews in Plant Science, 28(3):164-178, 2009., Darrell W Nelson and Lee E Sommers. Methods of soil analysis: Part 3 Chemical methods, 5:961-1010, 1996.). It relies on laboratory analysis through taking small-volume soil samples and oxidizing them at a high temperature. These procedures are expensive and time-consuming, thus not suitable for monitoring small soil carbon changes in the field and creating soil carbon maps of large areas (Umesh Acharya, et al., Carbon Management, 13(1):401-419, 2022.). There have been several in-situ methods, including spectroscopic methods and remote sensing, to provide faster, cheaper, and more accurate carbon measurement. Spectroscopic methods leverage the spectral behavior of soil car-bon, including near/mid-infrared spectroscopy (NIRS/MIRS), laser-induced breakdown spectroscopy (LIBS) and inelastic neutron scattering (INS). NIRS/MIRS detects carbon through the infrared reflectance in the near/mid infrared region that is affected by soil carbon (GW McCarty, et al., Soil Science Society of America Journal, 66(2):640-646, 2002). LIBS uses laser beam to form microplasma which emits light containing the spectral signature of carbon (David A Cremers, et al., Journal of Environmental Quality, 30(6):2202-2206, 2001., Michael H Ebinger, et al., Carbon sequestration in soils of Latin America. Food Products Press, New York, pages 407-422, 2006.). INS is based on the gamma rays emitted from the interaction of carbon nuclei and fast neutrons (Lucian Wielopolski, et al., Soil Science Society of America Journal, 72(5):1269-1277, 2008.). The high cost of these methods (over US$10k), however, limits their adoption (Amitava Chatterjee, et al., Critical Reviews in Plant Science, 28(3):164-178, 2009.).

### Soil Carbon, Soil Cover, and RF Signals

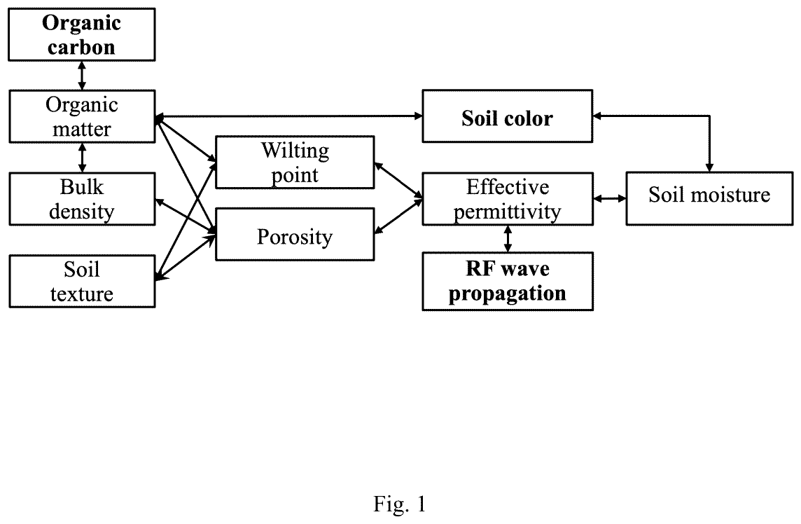

FIG. 1 illustrates the overall connection among soil carbon, soil color, and RF wave propagation in soil, interlinked through a series of soil physical properties (FIG. 1). Next, the relationships are introduced in detail.

### Relationship Between Soil Carbon and Soil Color

Soil organic carbon is one of the major pigments of soil color. There has been a long history of using soil color to determine soil carbon content. Traditionally, Munsell color charts have been used to determine soil carbon content. Such a subjective method involves substantial errors (Albert Henry Munsell. A Color Notation: A Measured Color System Based on the Three Qualities, Hue, Values and Chroma with Illustrative Models, Charts and a Course of Study Arranged for Teachers. Geo. H. Ellis, 1907.). Recent studies use chroma meters to get more accurate soil color measurement (Naoki Moritsuka, et al., Soil science and plant nutrition, 60(4):475-485, 2014., RA Viscarra Rossel, et al., Biosystems engineering, 100(2):149-159, 2008.). Statistically, there exists a negative relationship between soil color and soil carbon content. That is, the higher the carbon content, the darker the soil color. However, to ensure the accuracy of soil color measurement, the soil sample must be sieved, and its soil moisture needs to be controlled because soil moisture also changes soil color. Existing methods require either drying out or saturating the soil sample before performing the color measurement. Besides soil moisture, soil texture and iron oxides also influence soil color. Their impacts need to be calibrated when using soil color to determine carbon content.

### Relationship Between Soil Carbon and RF Wave Propagation

The propagation of RF signals in soil depends on the soil's dielectric permittivity: it slows down in soil because soil has a higher dielectric permittivity than air. Soil's dielectric permittivity, on the other hand, mostly depends on the amount of water in the soil. Therefore, various dielectric-based commodity soil sensors, including RF-based approaches such as GPR and TDR, measure soil permittivity to estimate soil moisture (Johan Alexander Huisman, et al., Vadose zone journal, 2(4):476-491, 2003., Kosuke Noborio. Computers and electronics in agriculture, 31(3):213-237, 2001.).

### Mapping Permittivity to Soil Moisture

The mapping from permittivity to moisture requires modeling permittivity as a function of soil properties. A widely adopted modeling approach is using mixing models which are empirically derived as third-order functions of soil volumetric water content (VWC), e.g., Topp's equation (G Clarke Topp, et al., Water resources research, 16(3):574-582, 1980.). The polynomial coefficients in the functions need to be adapted for different types of soils through extensive experimental data. These models are widely adopted by commodity dielectric-based soil moisture sensors to convert permittivity to VWC.

Another common modeling approach is using soil dielectric mixing models, which consider soil texture and therefore can be applied for different soil types. These models typically involve the impacts of soil texture, wilting point, porosity, VWC and radio frequency on permittivity (James R Wang and Thomas J Schmugge. IEEE Transactions on Geoscience and Remote Sensing, (4):288-295, 1980., Myron C Dobson, et al., IEEE Transactions on geoscience and remote sensing, (1):35-46, 1985.).

### Soil Dielectric Mixing Model

Related soil properties are now described.

Dielectric permittivity is a material property that describes a material's ability to store electrical energy in an electric field. Relative permittivity (unitless) refers to the ratio of absolute dielectric permittivity to the free-space permittivity. Effective permittivity, also known as apparent permittivity, refers to the relative permittivity of the soil mixture measured in-situ. For the rest of this Example, permittivity is used to refer to effective permittivity for simplicity.

Soil texture is the method to classify soil into different soil types. It is expressed as the proportion of different-sized soil particles, i.e., sand, silt and clay, where sand is the largest and clay is the smallest.

Porosity is the ratio of pore space in the total volume of soil. It indicates the amount of water/air soil can hold.

Bulk density is the dry soil weight divided by its volume. It is inversely related to porosity.

Wilting point is the soil water content below which plants cannot extract water from soil.

Transition moisture marks the point at which the relationship between permittivity and VWC shifts from a gradual to a rapid increase. This phenomenon is attributed to the transformation of water from a bound to a free state within the soil. Bound water is tightly bound to soil particles and has a smaller dielectric permittivity than free water (James R Wang and Thomas J Schmugge. IEEE Transactions on Geoscience and Remote Sensing, (4):288-295, 1980.). Existing literature has demonstrated a strong correlation between transition moisture and wilting point (James R Wang and Thomas J Schmugge. IEEE Transactions on Geoscience and Remote Sensing, (4):288-295, 1980., Richard Wayne Newton. Texas A&M University, 1977.). Therefore, the two terms are usually used interchangeably.

Considering these soil properties, the effective permittivity of soil at different VWC levels can be described as a three-phase equation that has been validated to generalize for different soil types and soil properties:

$${ϵ}_{e} = \{ \begin{matrix}( 1 - p ) {ϵ}_{s} + ( p - w ) {ϵ}_{a} + w {ϵ}_{b} & w \leq {w}_{wp} \\ ( 1 - p ) {ϵ}_{s} + ( p - w ) {ϵ}_{a} + w ( \frac{p - w}{p - {w}_{wp}} {ϵ}_{b} + \frac{w - {w}_{wp}}{p - {w}_{wp}} {ϵ}_{f} ) & {w}_{wp} < w \leq p \\ ( 1 - w ) {ϵ}_{s} + w {ϵ}_{f} & w > p\end{matrix} \qquad ( 1 )$$

where p is the porosity, w is the VWC, w_{wp} is the VWC at wilting point, ∈ₛ, ∈ₐ, ∈_{b} and ∈_{f} are the permittivity of soil, air, bound water and free water, respectively (Chang-Hwan Park, et al., Remote Sensing, 9(7):732, 2017.). ∈ₛ can be computed from the volumetric ratios of sand, silt and clay, v_{sand}, vₛᵢₗₜ, and v_{clay}, and their permittivity, ∈_{sand}, ∈ₛᵢₗₜ and ∈_{clay}, given as: ∈ₛ=v_{sand}∈_{sand}+vₛᵢₗₜ∈ₛᵢₗₜ+v_{clay}∈_{clay}.

### Soil Carbon's Impact on Permittivity

Soil carbon is closely correlated with porosity, an important variable in the mixing model (Eq. 1, above), and thus has a significant impact on permittivity. Specifically, soils richer in carbon have lower bulk density, higher porosity, higher wilting point and can hold more water. These combined factors result in a lower soil permittivity and, consequently, a faster propagation speed of RF signals within the soil.

Soil carbon's impact on permittivity affects the accuracy of dielectric-based soil moisture sensors. Traditional soil mixing models that map permittivity to VWC are mostly developed for mineral soils with low carbon contents. Using them for carbon-rich soils could introduce errors in the estimated VWC. To calibrate the impact of soil carbon, some earlier studies suggest changing the polynomial coefficients in the empirical mixing models (G Clarke Topp, et al., Water resources research, 16(3):574-582, 1980., CH Roth, et al., Journal of Soil Science, 43(1):1-13, 1992.). Recent work using dielectric mixing models considers the impact of soil carbon either implicitly by using different bulk densities for soils with different carbon contents, or explicitly by including soil carbon in the dielectric mixing model (Valery Mironov and Igor Savin. Physics and Chemistry of the Earth, Parts A/B/C, 83:57-64, 2015., Chang-Hwan Park, et al., Vadose Zone Journal, 18(1):190036, 2019.).

### Design

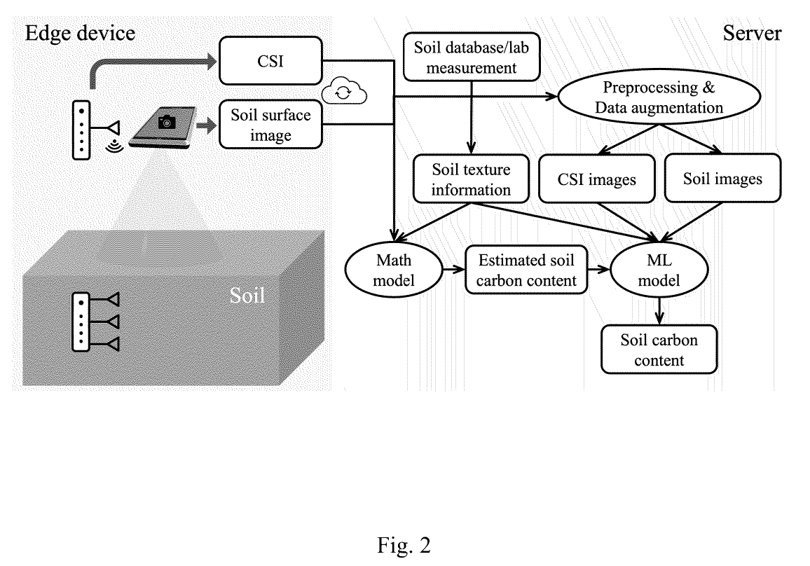

The design of Scarf is now described. As shown in FIG. 2, two RF nodes were used, one in the air as the transmitter and another in the soil as the receiver to collect channel state information (CSI) that correlates with soil properties. Meanwhile, a smartphone camera takes pictures of the soil surface to capture soil's optical information. The collected CSI and soil surface images are sent to an edge or cloud server for further processing. A hybrid approach combining mathematical modeling and machine learning (ML) was employed to estimate soil carbon content. Initially, a mathematical model leverages soil images and CSI to generate a preliminary carbon estimation. Recognizing the limitations of mathematical models in fully capturing the complex relationships among soil properties, an ML model was introduced to predict the error between the initial estimate and ground truth. The predicted error was then added back to the mathematical result to produce the final soil carbon estimation. Before training the ML model, preprocessing on both CSI and soil surface image was performed. Due to the limited size of measurement data, data augmentation was also applied to increase dataset size. The ML model was trained on data containing a diverse range of soil moisture and carbon content levels for each soil type. Both the mathematical model and ML model require soil texture information, which can be obtained from existing soil database, or laboratory measurement (Soil Survey Staff. “web soil survey.” natural resources conservation service, us department of agriculture. 2014.). Scarf's edge to server communication can benefit from existing infrastructure for data-driven agriculture to achieve reliable and secure network connectivity (Deepak Vasisht, et al., In 14th USENIX Symposium on Networked Systems Design and Implementation (NSDI), pages 515-529, 2017.).

Sensing Soil Carbon with RF Signals and Soil Surface Images

The relationship between RF signals and soil carbon and the relationship between soil images are now explained and modeled. Why a single signal modality does not work is now discussed. Mathematical models were used to show that two signal modalities are enough to determine soil carbon.

### RF Signals Vs Soil Carbon

RF signals may be used to detect soil carbon levels. First, existing work on soil dielectric mixing models considers soil carbon's impact on permittivity, with a goal to calibrate out the impact and improve the accuracy of soil moisture estimation. This indicates that, conversely, by leveraging the relationship between soil carbon and permittivity, soil carbon can also be inferred from permittivity. Second, recent work has demonstrated that RF signals from low-cost devices, such as Wi-Fi chips, can measure soil permittivity, enabled by the phenomenon that RF signals travel slower in soil because it has a higher permittivity than air (Jian Ding and Ranveer Chandra. In The 25th Annual International Conference on Mobile Computing and Networking (MobiCom), pages 1-16. ACM, 2019., Colleen Josephson, et al., In ACM SIGCAS Conference on Computing and Sustainable Societies, pages 299-311, 2021., Zhaoxin Chang, et al., Proceedings of the ACM on Interactive, Mobile, Wearable and Ubiquitous Technologies, 6(2):1-27, 2022., Yuda Feng, et al., In Proceedings of the 20th ACM Conference on Embedded Networked Sensor Systems (SenSys), pages 421-434, 2022.).

### Modeling Permittivity as a Function of Soil Carbon and Moisture

The three-phase equation (Eq. 1, above), where the parameters can be divided into three groups, was started with. (i) Parameters independent of soil carbon and soil moisture include the permittivity of soil, air, bound water and free water, ∈ₛ, ∈ₐ, ∈_{b}, ∈_{f}. These parameters can be considered as constants during the measurements. ∈ₛ depends solely on soil texture/type and is a constant given a soil type. ∈ₐ is usually set to 1. ∈_{b} and ∈_{f} are functions of operating frequency and soil temperature. Equations in were applied to get ∈_{b} and ∈_{f} (Chang-Hwan Park, et al., Vadose Zone Journal, 18(1):190036, 2019.). (ii) Parameters dependent on soil carbon are wilting point w_{wp} and porosity p. (iii) Soil volumetric water content (VWC), w. For simplicity, Eq. 1 can be rewritten as:

∈ₑ=f_{∈}(w,w_{wp},p) (2)

Existing work models w_{wp} and p empirically. A linear model of wilting point was adopted herein, given as w_{wp}=0.0298+0.0890v_{clay}+0.01360c, where v_{clay} is the volumetric ratio of clay in soil and oc is the organic carbon content (%) (Jun Liu, et al., In 2013 IEEE International Geoscience and Remote Sensing Symposium-IGARSS, pages 13-16. IEEE, 2013.). The equation's parameters were determined using 12 soil types and organic carbon contents. Empirical models of porosity p and bulk density BD given in Park et al. were used, which have been evaluated with over 30 sites containing wide varieties of soil types and carbon contents (Chang-Hwan Park, et al., Vadose Zone Journal, 18(1):190036, 2019.). p was modeled as a non-linear function of oc, BD, and volumetric ratios of clay and slit in soil, v_{clay} and vₛₗᵢₜ, written as p=fp (oc, BD, v_{clay}, vₛₗᵢₜ). BD is negatively correlated with oc: BD=−0.067oc+1.2301. Given a soil type with known v_{clay} and vₛₗᵢₜ, both w_{wp} and p are only functions of oc, written as w_{wp}=f_{wp}(oc) and p=fp (oc). Hence, the permittivity in Eq. 2 is only a function of w and oc, given as:

∈ₑ=f_{∈}(w,f_{wp}(oc),fₚ(oc)=f_{∈}(w,oc) (3)

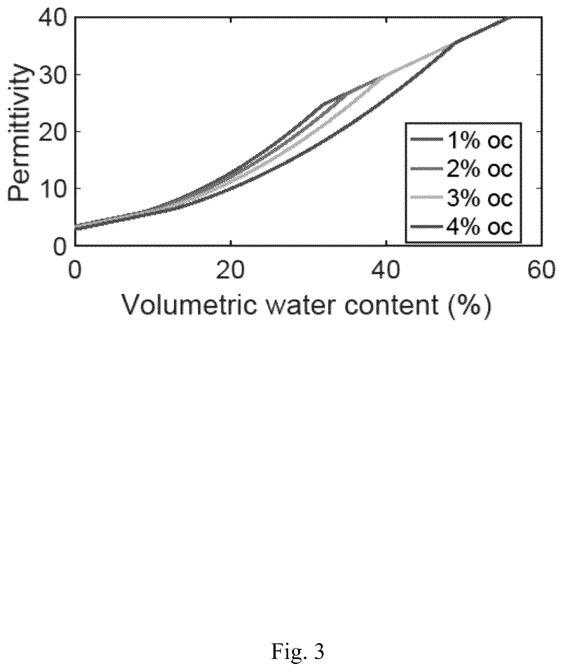

To quantify the impact of soil carbon on effective permittivity, simulations were first performed based on Eq. 3 (Eq. 3, above). FIG. 3 plots the simulated permittivity at 2.4 GHz Wi-Fi frequency band for soils with 40% clay, 40% slit and 20% sand, a composition used in the experiments (FIG. 3). The results indicated that given the same ground truth VWC, the permittivity decreases for soils with higher carbon contents. This decrease was significant enough to be observed when VWC falls in the range between wilting point and saturation point, e.g., 20-40% VWC. This behavior was also experimentally validated, and the results are discussed below.

Soil Surface Images Vs. Soil Carbon

Existing studies have shown that there is a negative relationship between soil carbon and soil lightness, typically modeled as a linear equation: L=−ax+b, where L is the lightness (unitless) in CIELAB color space, x is the organic carbon content oc or natural logarithm of oc, ln(oc), and a, b>0 are constants (Naoki Moritsuka, et al., Soil science and plant nutrition, 60(4):475-485, 2014., Umesh Acharya, et al., Carbon Management, 13(1):401-419, 2022.). However, existing studies use chroma meters with controlled light source and carefully prepared soil samples which are fine-grained and have controlled VWC.

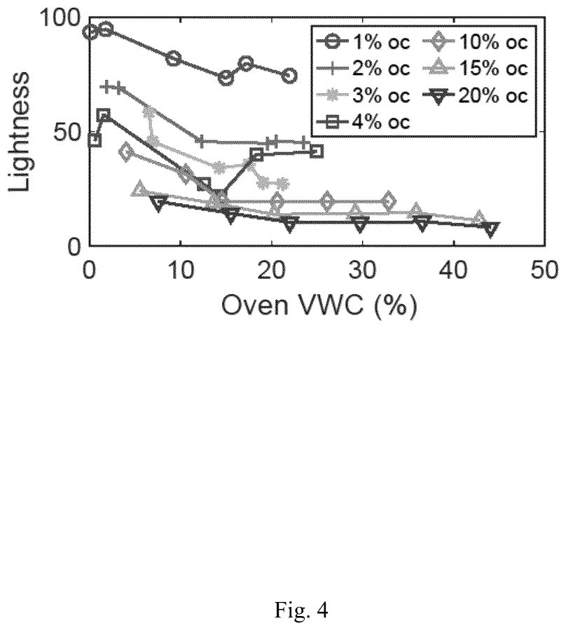

To demonstrate the possibility of using smartphone images to detect soil lightness change caused by soil carbon, soil surface images of soil boxes at different carbon and moisture levels were collected. These soil boxes are created with sand and compost manure for fine-grained control of soil carbon content. FIG. 4 plots the lightness computed from the soil surface images after calibrating ISO, exposure time, and aperture size of the smartphone camera (FIG. 4). It indicates a negative correlation between soil lightness and carbon content, consistent with previous studies. Notably, the speed of lightness decrease slowed down at higher carbon content. Additionally, lightness also decreased as VWC increased. The impact of soil moisture on soil lightness has also been identified in existing literature, while not explicitly modeled (S K Bhadra and M Bhavanarayana. Zeitschrift für Pflanzenernährung und Bodenkunde, 160(3):401-405, 1997., E A Paul, J A Shields, W K Head, R J St Arnaud, et al. Spectrophotometric measurement of soil color and its relationship to moisture and organic matter., DF Post, et al., Soil Science Society of America Journal, 64(3):1027-1034, 2000.).

### Modeling Lightness as a Function of Soil Carbon and Moisture

Based on the observations from FIG. 4, the model of lightness was derived as a function of oc and w:

L=−a ln(oc+1)+b−cw (4)

where a, b, c>0 are constants. The logarithm of oc was adopted because the decrease speed of lightness reduces as oc increases. ln(oc+1) was chosen instead of ln(oc) to avoid large negative values of ln(oc) when oc is close to 0.

a, b and c were determined empirically. Given a set of measured oc, w, and Z values, i.e., oc₁, oc₂, . . . , ocₙ, w₁, w₂, . . . , wₙ, and L₁, L₂, . . . , Lₙ, compute a, b and c can be computed by solving the following equation:

$${\underbrace{[ \begin{matrix}- ln ( {oc}_{1} + 1 ) & 1 & - {w}_{1} \\ - ln ( {oc}_{2} + 1 ) & 1 & - {w}_{2} \\ ⋮ & ⋮ & ⋮ \\ - ln ( {oc}_{n} + 1 ) & 1 & - {w}_{n}\end{matrix} ]}}_{A} {\underbrace{[ \begin{matrix}a \\ b \\ c\end{matrix} ]}}_{x} = {\underbrace{[ \begin{matrix}{L}_{1} \\ {L}_{2} \\ ⋮ \\ {L}_{N}\end{matrix} ]}}_{B} \qquad ( 5 )$$

It can be derived that x=(A^{T}A)⁻¹A^{T}B.

### A Single Signal Modality is not Enough.

FIG. 3 indicates that only knowing permittivity is not enough to determine soil carbon content, because a permittivity value can be mapped to different carbon values given different VWC values. Both permittivity and ground-truth VWC are needed to determine the carbon content. Similarly, as shown in FIG. 4, measuring soil lightness without knowing VWC is not also enough to determine carbon content, since a single lightness value can be mapped to different carbon values given different VWC values. The challenge is that measuring the VWC is very time-consuming. The ground truth oven drying method takes 24 hours per sample. To achieve rapid determination of soil carbon, combining RF signals with soil surface images was proposed.

### Combining the Two Signal Modalities to Determine Soil Carbon

It was identified that both permittivity and soil lightness are functions of soil carbon and soil moisture. A question here is: are the two signal modalities enough to estimate soil carbon? This is equal to a mathematical problem: given ∈ₑ and L obtained from RF signals and soil surface images, can a system consisting of Eq. 3 and Eq. 4 be solved, where oc and w are the only two unknown variables?

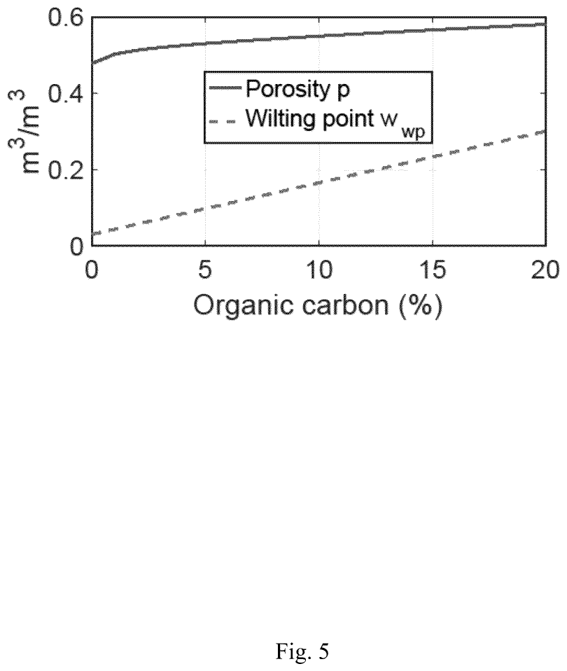

It was first shown that in both equations, there are monotonic relationships between oc and w. The monotonic relationship in Eq. 4 is obvious: by fixing L as a constant, w↑⇒oc↓. For Eq. 3, it was noticed that its variables depending on soil carbon, i.e., p and w_{wp}, both increase monotonically over oc, as shown in FIG. 5, based on the simulation for both field soils and sand compost mixtures. With ∈_{f}>∈_{b}>∈ₛ>∈ₐ as a known knowledge, the relationship between oc and w given a fixed ∈ₑ can be derived by analyzing the three phases in Eq. 1. (i) Phase 1: the equation can be rewritten as w∈_{b}+ (∈ₐ−∈ₛ)p+C=0, where C is a constant. Since (∈ₐ−∈ₛ)<0, oc↑⇒p↑⇒w↑. (ii) Phase 2: The equation can be rewritten as w[x(∈_{b}−∈_{f})+∈_{f}−1]+ (∈ₐ−∈ₛ)p+C=0, where x=(p−w)/(p−w_{wp}) and (∈_{b}−∈_{f})<0. Therefore, oc↑⇒p↑, w_{wp}↑⇒x↑⇒w↑. (iii) Phase 3: Here ∈ₑ only depends on w and increasing oc does not change w. Based on the three-phase analysis, it can be seen that as oc increases, w will first increase and then stay the same after w>p.

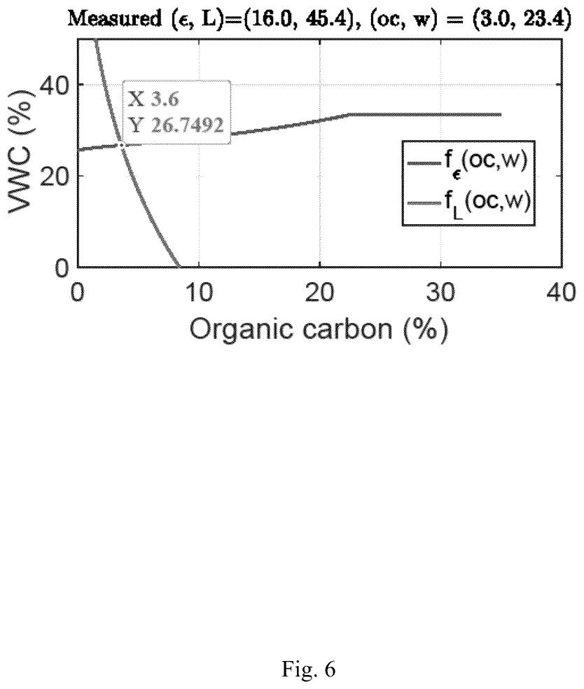

Given the monotonic relationships between oc and w in Eq. 3 and Eq. 4 and a pair of measured L and ∈ₑ, there exists at most one pair of oc and w that satisfies both equations. FIG. 6 plots an example of how to solve this problem. Given a pair of L and ∈ₑ, the two equations can be plotted as two non-parallel curves in the figure. The two curves have an intersection that corresponds to the solution of oc and w.

To automate the search of the intersection, an optimization problem was formulated. Eq. 3 and Eq. 4 were combined through substitution to get an equation with a single unknown variable: ∈ₑ=fₑ([−aLn(oc+1)+b−L]/c, oc). The goal was to find an oc that minimizes the following objective function:

f_{obj}=|ε̅ₑ−f_{∈}[−aLn(oc+1)+b−L̅]/c,oc) (6)

where ∈̅ and L̅ are the measured effective permittivity and soil lightness. After obtaining oc, the corresponding w can be determined from Equation 4.

### Data Collection and Calibration

How Wi-Fi signals are leveraged to collect CSI, and the necessary calibration for soil surface images are introduced.

### CSI Collection

The goal was to capture CSI that contains permittivity information of soil, and use it to generate the input to the ML model. Strobe's approach that uses a single-antenna transmitter and a multi-antenna array to get soil-dependent CSI was adopted (Jian Ding and Ranveer Chandra. In The 25th Annual International Conference on Mobile Computing and Networking (MobiCom), pages 1-16. ACM, 2019.). Besides Strobe's horizontal placement of antennas (FIG. 7A), deploying the antennas vertically was also supported (FIG. 7B).

### Practical Use Cases

The two antenna deployment methods correspond to two use cases that do not introduce significant deployment effort. (i) The horizontal deployment can be used for long-term carbon monitoring with the underground RF node remaining buried, which only requires a one-time effort to dig up the soil and bury the antenna array. This is helpful to monitor long-term carbon changes, e.g., carbon loss after tillage, and carbon increase with carbon management practices like no tillage and straw mulch coverage. (ii) The vertical deployment makes Scarf portable for field measurements at different locations. The antennas can be plugged into soil in a similar way as commodity soil sensors, without the need to dig up a large volume of soil.

CSI Vs. Permittivity

In both setups, the relative time-of-flight (ToF) of a signal propagating to two adjacent antennas can be simplified to Δt=nΔy/c, where Δy is the distance between antennas, c is the speed of light, and n is the refractive index. n can be related to the effective permittivity ∈ₑ as n=√∈ₑ, so Δt=Δy√∈ₑ/c. This relative ToF corresponds to a phase rotation of −2πfΔt, where f is the carrier frequency. In a multipath environment, CSI is the sum of multiple paths, where only the shortest path contains the phase rotation of interest. First pre-processing steps were performed including noise reduction and ToF sanitization [56], and then a multipath resolving technique, MUSIC [57], was applied to extract the shortest path and estimate Ee (Manikanta Kotaru, et al., In Proceedings of the 2015 ACM Conference on Special Interest Group on Data Communication (SIGCOMM), pages 269-282, 2015., Ralph Schmidt. IEEE transactions on antennas and propagation, 34(3):276-280, 1986.).

### Image Calibration

The lightness of image depends on scene luminance and camera settings. Prior work [58] demonstrates that a digital camera can measure luminance after calibrating camera settings including ISO, exposure time and aperture area. A pixel's digital number correlates with the scene luminance and camera settings as follows:

N∝tSLₛ/f² (7)

where t is the exposure time, S is the ISO, Lₛ is the scene luminance, and f is the aperture number. The settings of t, S and f are accessible from the metadata of images taken by smartphones. Reference settings of t, S and f were chosen, e.g., 1/120, 100 and 1.8, and every image was calibrated to the reference leveraging Eq. 7. Since experiments were conducted under the same scene luminance, Ls was not explicitly calibrated. When there is a change in scene luminance, Ls can be calibrated by taking a picture of a reference object, e.g., a white paper. In addition to lighting conditions, a more comprehensive approach should also consider factors like camera makes and models. Recent research has demonstrated the potential of deep learning-based image normalization for calibrating these factors. Advanced image calibration techniques may be investigated (Neha Sharma, et al., In Proceedings of the 29th Annual International Conference on Mobile Computing and Networking (MobiCom), pages 1-15, 2023.).

### Environmental Variations

Four types of variations were considered. (i) Antenna misalignment. In real-world conditions, it is hard to perfectly align the transmitter and receiver antennas. To reduce the impact of misalignment, for each soil sample, multiple CSI samples were collected by moving or rotating the transmitter, and were used for ML model training and testing. (ii) Soil surface disruptions. These impacts are reduced by falttening the soil surface and removing objects on it before conducting measurements. (iii) Small-scale spatial variations. Scarf measures the average VWC over a path length of around 10 cm in soil, which smooths out small-area variations in VWC as well as the impact of non-soil objects in soil (Jian Ding and Ranveer Chandra. In The 25th Annual International Conference on Mobile Computing and Networking (MobiCom), pages 1-16. ACM, 2019., Zhaoxin Chang, et al., Proceedings of the ACM on Interactive, Mobile, Wearable and Ubiquitous Technologies, 6(2):1-27, 2022.). Similarly, soil surface images were averaged across a region. (iv) VWC Variations across different locations and times. VWC Variations were taken care of by the mathematical models, which utilize CSI and image measured at a location and time to estimate VWC while simultaneously decoupling its influence on carbon estimation.

### Applying ML to Improve Accuracy

The mathematical models presented herein capture the major relationships between soil carbon, moisture, permittivity and lightness. However, they may have missed some underlying relationships which introduce deviations between mathematical results and the ground truth. To handle the hidden relationships, applying ML was proposed.

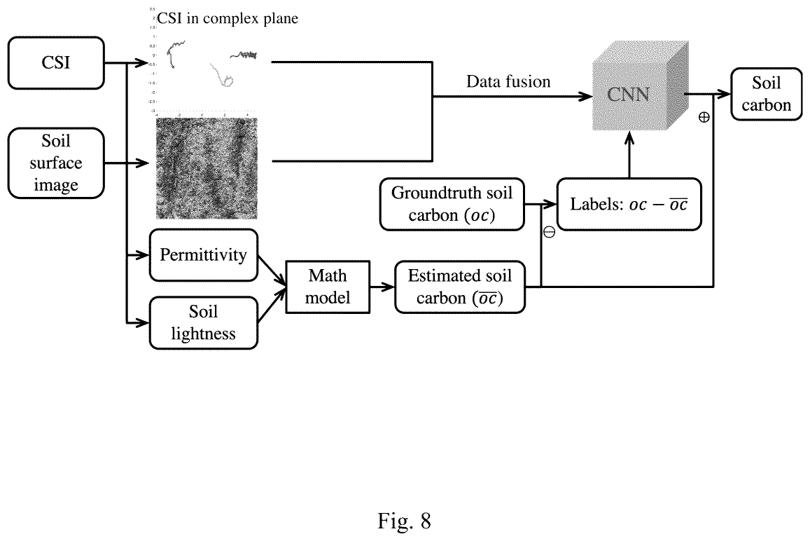

FIG. 8 shows an overview of the ML model design for improving math-computed soil carbon. A set of CSIs and soil images were first used to compute soil permittivity and soil lightness. The results were fed into the mathematical model (Eq. 6) to perform an initial estimation of soil carbon content, oc. The labels were then generated for the ML model by subtracting oc from the ground truth oc obtained with standard laboratory measurement. For the input to the ML model, an early fusion approach was considered to combine the two signal modalities before they enter the feature extraction network. Any model architecture may be used to handle multi-modal signals. For feature extraction, one of the most popular convolutional neural networks (CNNs), ResNet, was adopted (Kaiming He, et al., In Proceedings of the IEEE conference on computer vision and pattern recognition (CVPR), pages 770-778, 2016.). The feature extraction network takes the fused CSI and image data and labels as input to predict the error between the math-computed soil carbon content and the ground truth. In the end, the predicted error was added back to the mathematical result to produce the final soil carbon estimation.

### Generating Input to ML Model

Pre-processing and data augmentation were performed for CSA and images.

CSI. The goal here was to generate input that retains information as much as possible and can be interpreted by the ML model. A CSI sample is a matrix of complex numbers, with a size of 3×330 in the experiments. It was converted to a CSI image by plotting the CSI in the complex plane to keep both phase and amplitude information. An alternative CSI representation is the MUSIC spectrum presented as a relative ToF-absolute ToF heatmap. It is an intermediate result of the signal processing algorithms in prior work (Jian Ding and Ranveer Chandra. In The 25th Annual International Conference on Mobile Computing and Networking (MobiCom), pages 1-16. ACM, 2019., Manikanta Kotaru, et al., In Proceedings of the 2015 ACM Conference on Special Interest Group on Data Communication (SIGCOMM), pages 269-282, 2015.). The experimental results indicates that its performance is similar to the CSI image.

Soil surface images. A smartphone was used to take images of soil surface for each carbon level and moisture level. The original images have a size of 4032×3024. In order to reduce computation overhead and in the meantime capture enough information, e.g., color and texture, each image was cropped to a size of 448×448 and then resized with a factor of 0.5 to create a 224×224 RGB image. The size of 224×224 was chosen because it is a commonly adopted input image size for CNNs such as ResNet (Kaiming He, et al., In Proceedings of the IEEE conference on computer vision and pattern recognition (CVPR), pages 770-778, 2016.).

Data augmentation. To reduce overfitting, data augmentation was applied to both CSI and images to generate multiple images for a soil surface. Data augmentation is a widely adopted technique in computer vision to expand dataset size and reduce overfitting. For CSI, two methods for data augmentation were applied. First, two measured CSI samples were interpolated to produce a new sample. Second, additive white Gaussian noise (AWGN) and random relative phase shift were added among antennas, which emulates the case where soil has variations that lead to relative phase variations. For images, images were first randomly rotated and then cropping of a random region was performed. Common approaches for data augmentation in computer vision also include brightness and color adjustments. Brightness and color adjustments were not adopted since these are useful information correlated with soil carbon contents.

### Implementation

Hardware. Scarf's CSI collection component was implemented with WARP boards (Rice University. Warp project. http://warpproject(dot)org.). The transmitter antenna was connected to one board and the receiver antenna array to another board though cables. For both deployments in FIG. 7, the WARP boards are not buried. Practically, the horizontal deployment requires the antenna array to remain buried. Its connected WARP board may be replaced with a Wi-Fi chipset that can be buried together with the array. The radio frequency was swept across all available 2.4 GHz channels to collect CSI of the entire available 70 MHz bandwidth at 2.4 GHz Wi-Fi spectrum. The necessary calibration steps were performed to remove phase offsets and distortions caused by hardware impairments, same as those described in Ding et al. (Jian Ding and Ranveer Chandra. In The 25th Annual International Conference on Mobile Computing and Networking (MobiCom), pages 1-16. ACM, 2019.).

Software. The mathematical models, CSI and image pre-processing, and data augmentation were implemented in MATLAB. To support data fusion of two signal modalities, a customized ResNet regression model with Pytorch, a widely used deep learning library, was implemented. Due to a relatively small dataset, a SGD optimizer, a small batch size of 16, and 14 ResNet layers were used (Adam Paszke, et al., Advances in neural information processing systems, 32, 2019). Mean squared error (MSE) was used as the loss function, which was effective to improve R² during training. All ResNet training and testing were run on an NVIDIA GeForce RTX 4070 GPU.

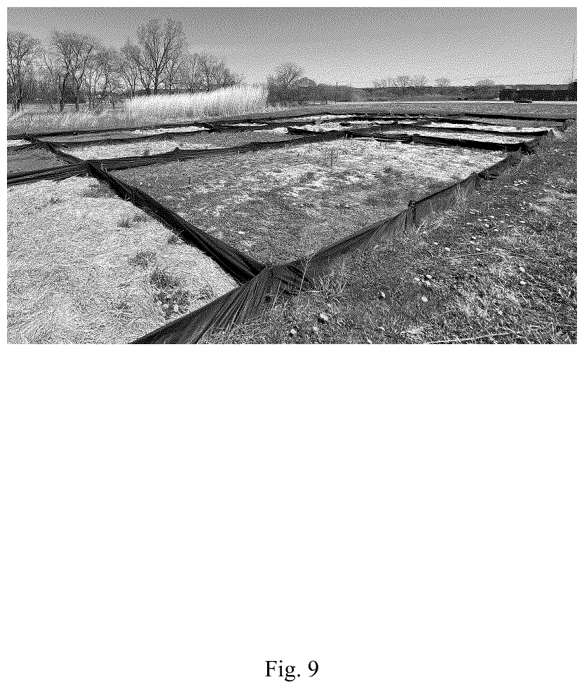

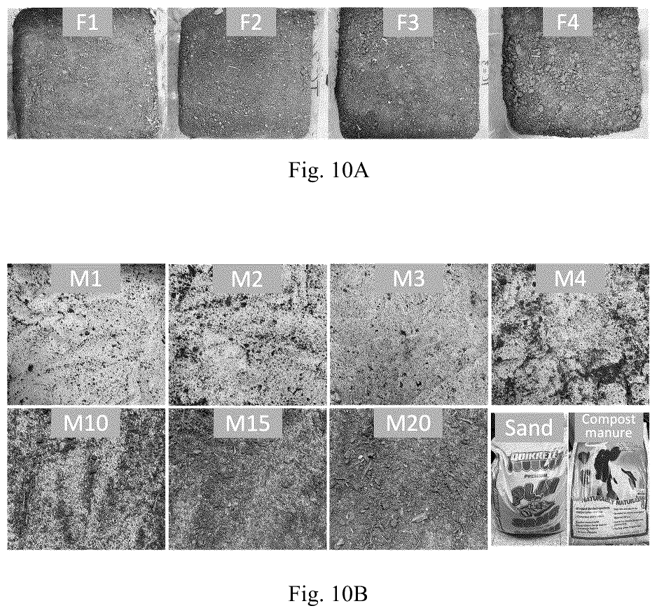

Experimental Setups. To test real soils, soils maintained in the field at different carbon contents (FIG. 9) were used to create four soil boxes with 1-4% carbon contents, as shown in FIG. 10A. They were labeled as F1-F4. These soils consisted of 40% clay, 40% silt and 20% sand. Since it is hard to precisely control carbon contents in real soils, sand compost mixtures were also created by mixing sand and compost manure. By controlling the percentage of sand and compost manure, boxes at 1-4%, 10%, 15% and 20% carbon contents were created, as shown in FIG. 10B. These mixtures were labeled as M1-M20.

### Evaluation

First, laboratory analysis of soil properties were performed for the field soils and sand compost mixtures used in the experiments to get ground truth knowledge of soil carbon's impact on soil properties. Them, the permittivity measured by Scarf was evaluated and compared to commodity soil sensors to validate that Scarf can detect soil carbon's impact on permittivity. Finally, the performance of the mathematical model and ML model was evaluated. Since the results for horizontal and vertical antenna deployments were similar, results were mainly reported for the horizontal deployment (FIG. 7A).

Laboratory analysis of soil properties. Some important soil properties were characterized, which were used as ground truths to demonstrate that the soil and sand compost mixture boxes that were created have different carbon contents, leading to different bulk densities and water retention curves. The methods used to measure these properties are first explained. For each of the measurements, 3 soil samples were taken and the average was computed to get more accurate results.

VWC. Gravimetric water content (GWC) was first measured with an oven drying method, calculated as (weight of wet soil−weight of dry soil)/(weight of dry soil), and then GWC was multiplied by bulk density of soil to get VWC. Each soil sample was dried in an oven at 105 °C for 24 hours.

Soil organic carbon. The standard dry combustion method was used to measure soil organic carbon (Darrell W Nelson and Lee E Sommers. Methods of soil analysis: Part 3 Chemical methods, 5:961-1010, 1996). 10-15 mg of 250-μm sieved soil samples were takin in tin capsules and analyzed using a Flash 2000 NC soil analyzer.

Bulk density. The standard core method was used (R B Grossman and T G Reinsch. Methods of soil analysis: Part 4 physical methods, 5:201-228, 2002.). Soil was sampled in a cylindrical core and then the soil sample's weight and VWC were measured. Bulk density was calculated as (weight of dry soil/the volume of the core).

Water retention. VWC was measured at −33 to −1500 kPa pressures with Pressure Plate Extractors (Arnold Klute. Methods of soil analysis: part 1 physical and mineralogical methods, 5:635-662, 1986.). A pressure in the range of 10-33 kPa was used to determine the soil's water holding capability against gravity, while a pressure of −1500 kPa was used to determine the wilting point.

Commodity soil sensors. To verify the drop of effective permittivity over soil carbon, Scarf was compared against 2 commodity soil sensors, i.e., Meter TEROS 10 and DecagonGS3. Meter TEROS 10 measures VWC and raw reading in mV, which can be converted to effective permittivity using the equation in its manual (METER Group. TEROS 10 user manual. 2021.). Decagon GS3 measures effective permittivity, EC and temperature.

### Microbenchmarks

Laboratory soil analysis results. The ground truth soil properties were first looked at.

Soil organic carbon and bulk density. The soil organic carbon and bulk density for field soils, pure sand, pure compost manure, and the mixtures of sand and compost manure were analyzed. The results are shown in Table 3 (Table 3, below). The sand compost mixtures were created based on the carbon content of pure compost manure and by controlling the relative weight of sand and compost manure. As can be seen, the measured carbon contents of field soils were close to the expected values, while the carbon contents of sand compost mixture boxes were slightly higher than the expected values. A possible reason is that the estimated carbon content for pure compost manure is lower than its actual carbon content. This could happen since small samples are taken for carbon analysis and these samples may be biased. These measured values were used for the rest of the evaluation. In addition, a negative relationship was observed between bulk density and carbon content, which is consistent with earlier discussion in this Example.

TABLE 3
Laboratory results of soil carbon and bulk density
for field soils (F1-F4), sand, compost manure,
and their mixtures (M1-M4, M10, M15, M20).

| Soil type | Carbon (%) | Bulk density (Mg/m³) |
|---|---|---|
| F1 | 1.35 | 1.038 |
| F2 | 2.30 | 1.020 |
| F3 | 3.15 | 1.016 |
| F4 | 3.88 | 0.983 |
| Sand | 0 | 1.458 |
| Compost manure | 47.66 | 0.178 |
| M1 | 1.54 | 1.043 |
| M2 | 3.03 | 0.966 |
| M3 | 4.25 | 0.893 |
| M4 | 4.90 | 0.889 |
| M10 | 14.14 | 0.792 |
| M15 | 18.97 | 0.718 |
| M20 | 31.58 | 0.601 |

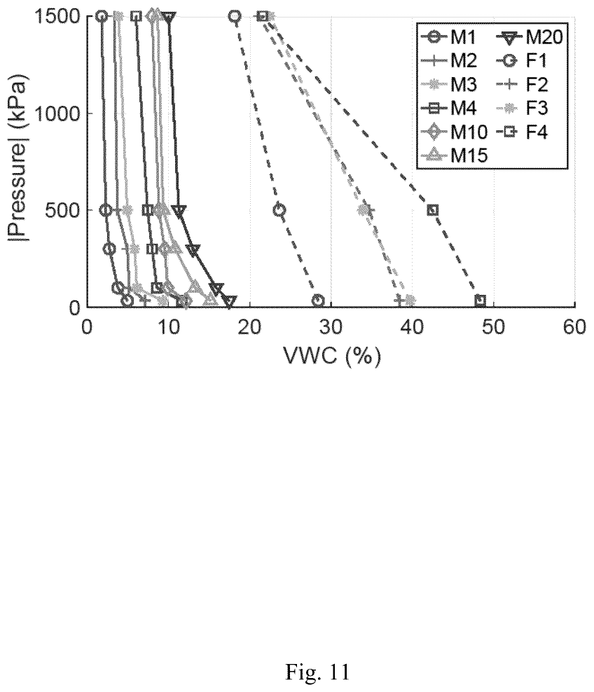

Water retention curves. FIG. 11 plots the water retention curves. Two trends were observed that met our expectations. First, field soils had higher VWC than sand compost mixtures. This is because the field soils contain 40% clay, which helps retain more water. Second, given a pressure level, for both soil types, VWC increased over carbon content. Moreover, the increase in wilting point (i.e., −1500 kPa) over carbon content was consistent with the observation in FIG. 5.

Soil carbon's impact on RF signals. To verify that RF signals can detect permittivity changes over soil carbon content, experiments were conducted with soils containing different carbon and moisture contents. The top layer of soil (0-10 cm), where soil carbon is concentrated in practice, was measured. Permittivity was estimated from collected CSIs with MUSIC-based data processing algorithm. For each soil box, the VWC was varied by adding tap water and mixing water with soil evenly. For each moisture level, permittivity was measured with Scarf, Meter TEROS 10 and Decagon GS3. The VWC was measured by an oven drying method as the ground truth.

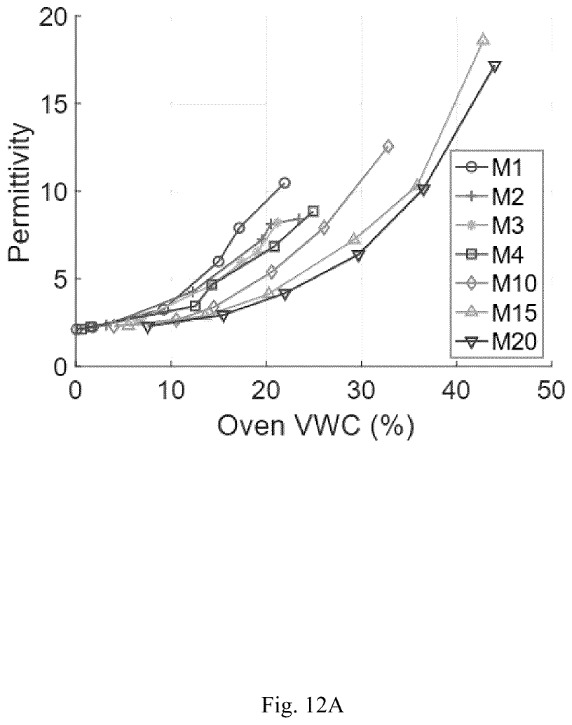

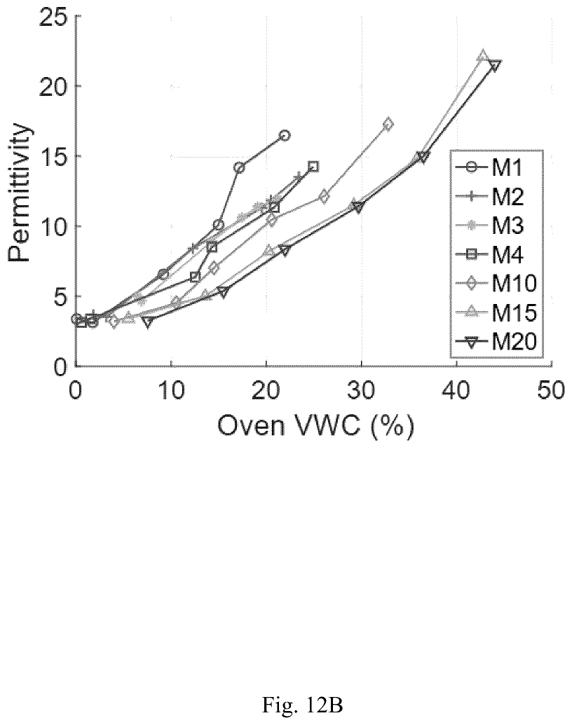

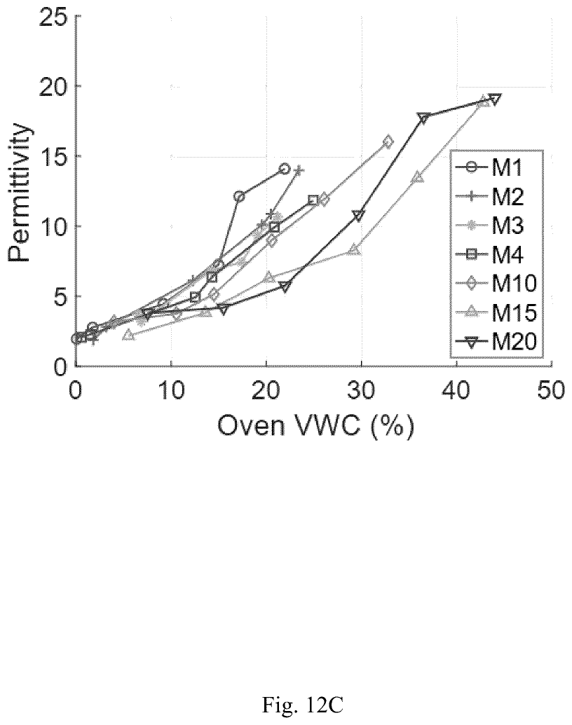

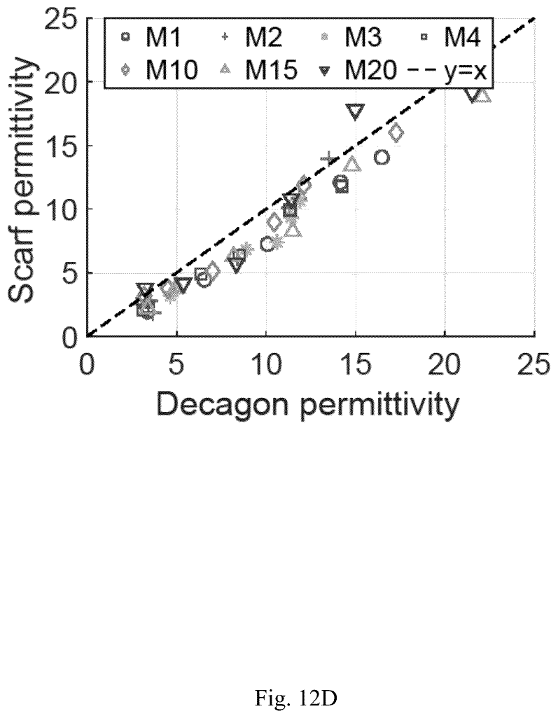

Sand compost mixtures. FIG. 12 plots the permittivity of sand compost mixtures measured by different devices. For all three devices, the general trends were the same: higher carbon content resulted in lower permittivity under the same ground truth VWC. It was noticed that Scarf had slightly higher variations. There are several possible reasons, e.g., multipath may not be fully removed during data processing, and different amounts of force on the soil surface may have been applied during antenna deployment. FIG. 12D shows permittivity results obtained from Scarf exhibit a strong correlation with those acquired from the Decagon sensor. Similarly, Scarf's results also demonstrated a high correlation with the Meter sensor. The correlation was 0.97 for Scarf and the Decagon sensor, and 0.95 for Scarf and the Meter sensor.

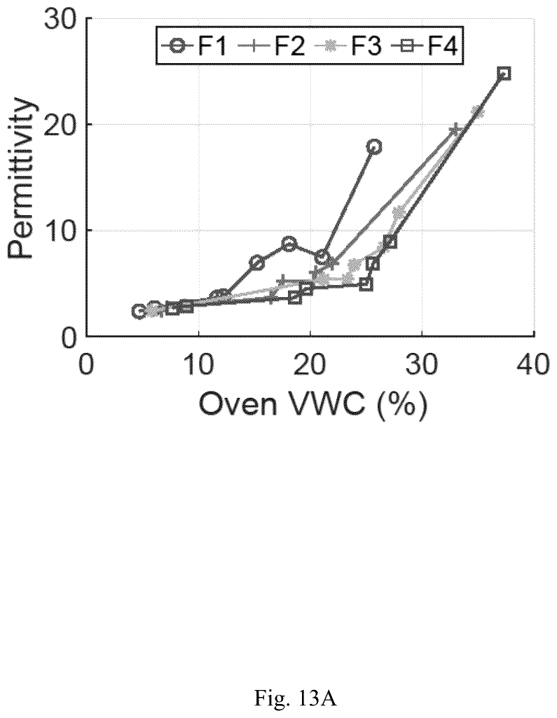

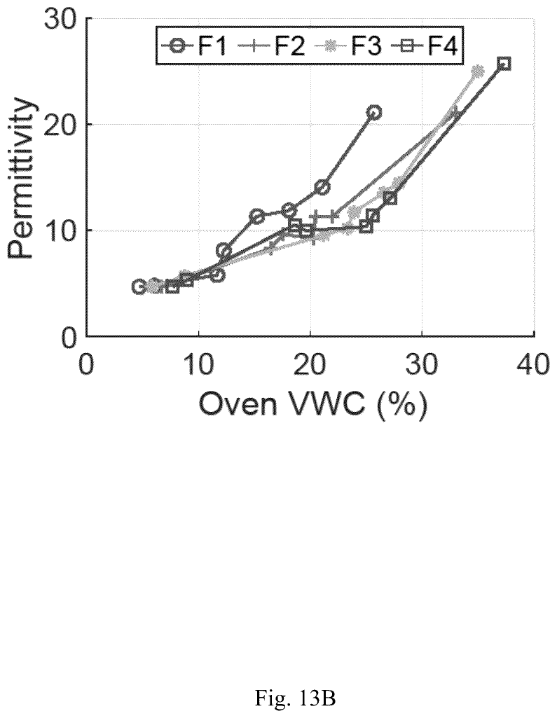

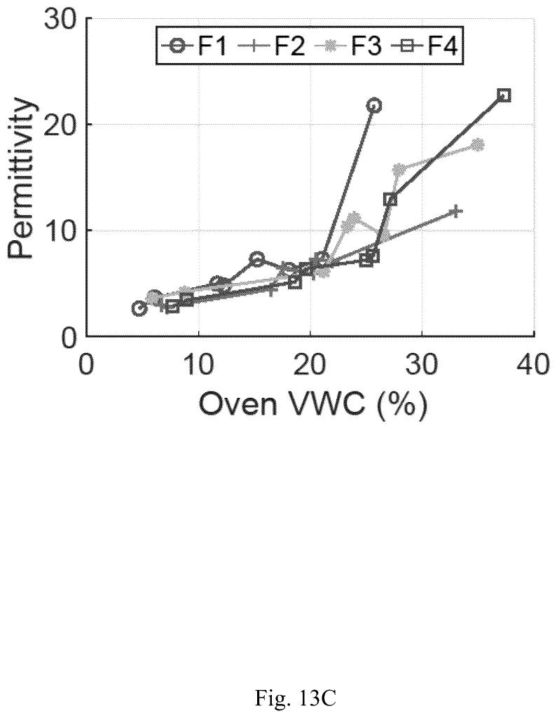

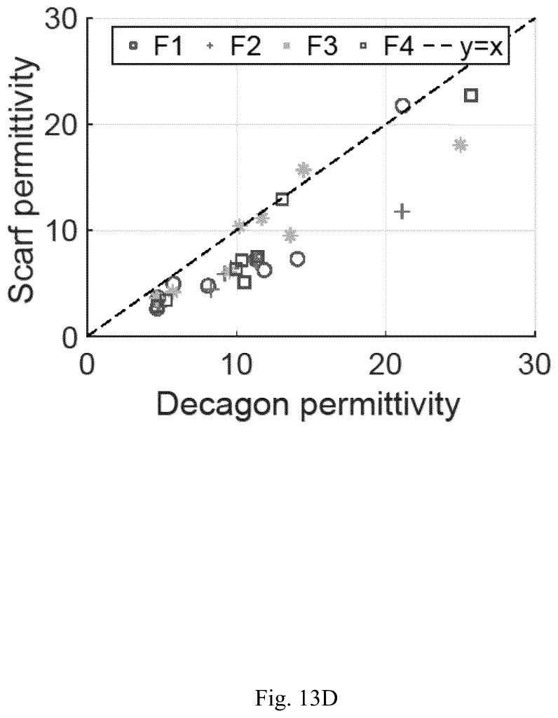

Field soils. FIG. 13 plots the permittivity of field soils measured by different devices. It was noticed that Meter and Decagon can detected the permittivity decrease for F1-F2. However, the curves of F2-F4 overlap more. This might have been caused by the close carbon contents between F2-F4, as shown in Table 3 (Table 3, above). Similar to the case of sand compost mixtures, it was also observed that Scarf's results had more variations than the Meter and Decagon sensors. FIG. 13D shows the correlation of permittivity results obtained from Scarf and the Decagon sensor. Compared to sand compost mixtures, the correlation here was slightly worse, which dropped to 0.91.

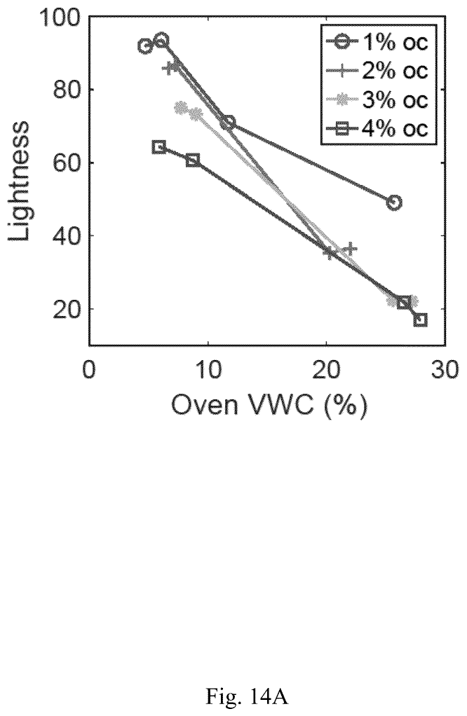

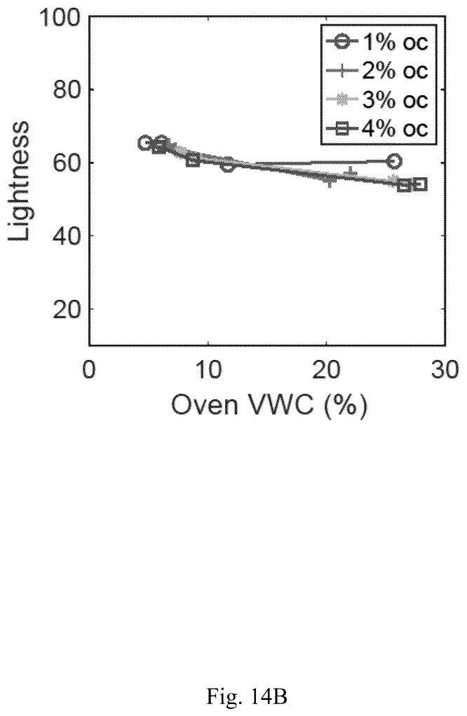

Soil carbon's impact on soil lightness. For sand compost mixtures, the trend of soil lightness's decrease over soil carbon and moisture was previously discussed in (FIG. 4). For field soils, the lightness results of smartphone images after calibrating camera settings are shown in FIG. 14A. The general trends were the same as those of sand compost mixtures. However, the decrease in lightness over soil carbon content was smaller for field soils, suggesting that parameters in Eq. 4 needed adjustment for different soil types. By comparing FIG. 14A and FIG. 14B, the importance of image calibration can also be seen, without which the lightness results of different soil carbon contents are overlapped and do not decrease much over VWC.

### Soil Carbon Sensing Performance

Next, the soil carbon sensing performance of the proposed mathematical model and the describe ML was evaluated.

Performance metrics. Correlation coefficient, co-efficient of determination, and mean squared error, which are commonly used metrics, were used for evaluating model fitting performance. Correlation coefficient is defined as

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} \sqrt{{\sum }_{i = 1}^{n} {( {y}_{i} - \overline{y} )}^{2}}}} \qquad ( 8 )$$

where n is the size of the testing dataset, X={x₁, x₂, . . . , xₙ} is the set of predicted carbon labels and Y={y₁, y₂, . . . , yₙ} is the set of ground truth carbon labels, and x and y are the mean values

$${R}^{2} = 1 - {\sum }_{i = 1}^{n} {( {y}_{i} - {x}_{i} )}^{2} / {\sum }_{i = 1}^{n} {( {y}_{i} - \overline{y} )}^{2} .$$

The range of R² is (−∞, 1]. For both r and R², a value closer to 1 represents a better performance. The mean squared is given as

$$M S E = {\sum }_{i = 1}^{n} {( {y}_{i} - {x}_{i} )}^{2} / n .$$

Mathematical model. CSI and soil surface images were collected of 7 sand compost mixture boxes each with 6 moisture levels, and 4 field soil boxes each with 6 moisture levels. At each moisture level, CSIs were collected at 3-6 transmitter locations by moving or rotating the transmitter, and averaging the estimated permittivity. 42 data points for sand compost mixtures and 24 data points for field soils were acquired. First, lightness estimated from soil surface images was used to find parameters for Eq. 4. Then, Eq. 6 was used to compute soil carbon content and VWC. It is worth noting that oven-based VWC w was used to estimate the constants in Eq. 4. The experimental results suggested that w can be replaced by VWC estimated from RF signals without sacrificing performance, simplifying the process of obtaining the lightness model.

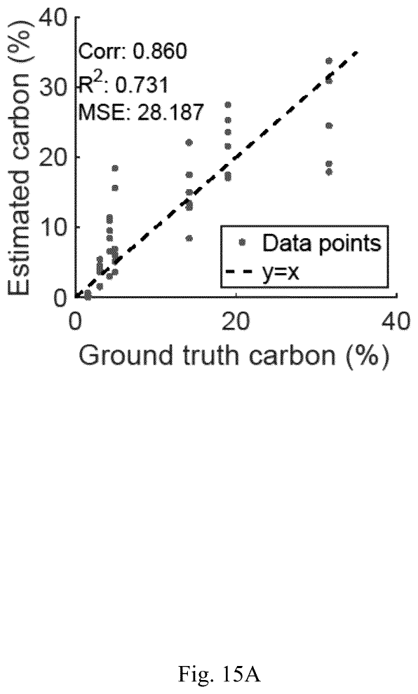

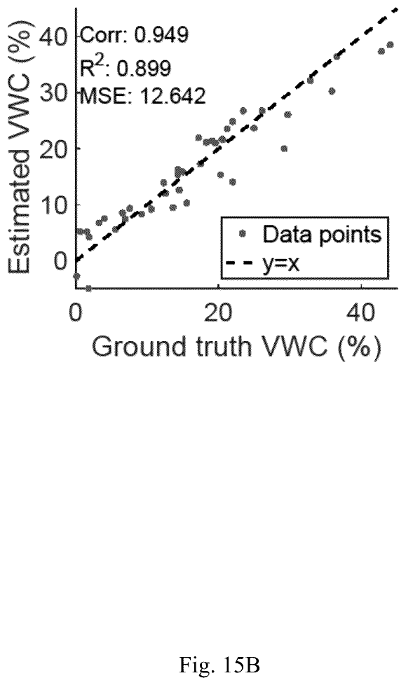

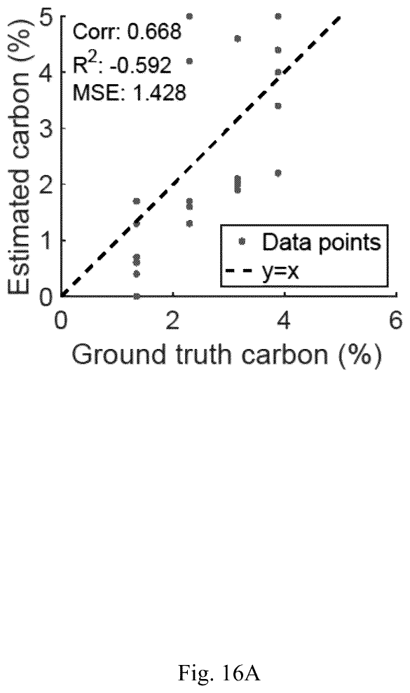

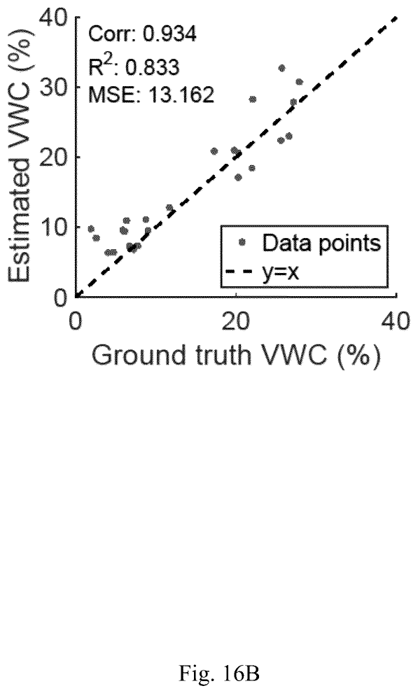

FIG. 15 and FIG. 16 present the results computed by the mathematical model for the two soil types. Both soils have superior performance in VWC estimation. Sand compost mixtures had better correlation and R² of carbon estimation than the field soils because they have a larger dataset and a larger range of carbon contents. With only 24 datapoints tested for field soils, a single outlier can significantly degrade the performance of correlation and R². The MSE of field soils, however, was much smaller than that of sand compost mixtures, meaning that the carbon estimation errors of field soils were reasonably small. Overall, it can be concluded that the mathematical model is a good fit for the experimental data, while some relationships leading to the carbon estimation errors may not be fully captured.

ML model. Datasets. 213 samples for the sand compost mixtures and 110 samples for the field soils were collected experimentally. Data augmentation was performed on these samples as described herein. After data augmentation, 672 samples for the sand compost mixtures and 384 samples for the field soils were obtained. To avoid model overfitting, training and testing datasets were split to ensure that the moisture level and carbon content combination of all the testing samples had not been seen from the training dataset. Sand compost mixtures and field soils were trained and tested separately.

Performance of leaving some moisture levels out. First, the ML model performance was tested when some moisture levels are not seen during training. For sand compost mixtures, there are 42 different moisture levels of all boxes, from which samples of 11 moisture levels were randomly picked to create the testing dataset. For field soils, 4 out of 24 moisture levels were picked as the testing dataset. These samples were also excluded from the parameter calculation in Eq. 5, so they were unseen in the mathematical model. The results were averaged over 5 random samplings of soil moisture levels. As shown in Table 4, for both soil types, the proposed method of combining mathematical model with ML can significant improve the performance of only using a single model (Table 4, below). It was observed that during the training process, the Math&ML model can effectively reduce the training loss of predicting the error between math-computed carbon and ground truth. The correlation and R² results of field soils appeared to be worse than sand compost mixtures because the field soils had a smaller range of carbon content. Interestingly, the performance of solely using the ML model is the worst for field soils, possibly due to the limited dataset size. In contrast, the Math&ML model demonstrated superior performance with the same dataset, emphasizing the value of integrating domain-specific knowledge, i.e., the mathematical model, into ML training.

TABLE 4
Performance of soil carbon determination.

| Soil | | | | |
|---|---|---|---|---|
| type | Model | r | R² | MSE |
| Sand | Math | 0.896 ± 0.044 | 0.784 ± 0.105 | 20.788 ± 14.297 |
| Sand | ML | 0.947 ± 0.039 | 0.842 ± 0.148 | 14.116 ± 12.584 |
| Sand | Math&ML | 0.963 ± 0.027 | 0.911 ± 0.070 | 8.491 ± 7.997 |
| Field | Math | 0.863 ± 0.078 | 0.195 ± 0.398 | 0.772 ± 0.357 |
| soil | | | | |
| Field | ML | 0.431 ± 0.341 | 0.179 ± 0.265 | 0.736 ± 0.238 |
| soil | | | | |
| Field | Math&ML | 0.880 ± 0.063 | 0.417 ± 0.269 | 0.523 ± 0.242 |
| soil | | | | |

Horizontal vs. vertical antenna deployment. The results in Table 2 are based on the horizontal deployment (FIG. 7A, Table 2, above). Data were also collected for the vertical deployment (FIG. 7B). Following the same data processing procedures, the average r, R², and MSE of the vertical deployment were 0.967, 0.923, and 7.177, respectively, for sand compost mixtures. The slightly better performance of the vertical deployment could be because the environmental noises are less when the antennas are all plugged into soil.

A discussion of the example is now described.

Increasing the practicality of Scarf. Scarf is a proof-of-concept design and implementation that uses invasive antenna deployment to sense soil carbon, limiting its practical applications. Scarf may be transitioned to a non-invasive system. However, a significant gap still exists between the cost-effectiveness and non-invasiveness of the RF-based techniques given in Table 1 and the invasive version of Scarf. It is also feasible to develop a non-invasive carbon sensing system by integrating a non-invasive RF system like COMEt into Scarf (Usman Mahmood Khan and Muhammad Shahzad. In Proceedings of the 28th Annual International Conference on Mobile Computing and Networking (MobiCom), pages 242-254, 2022.). However, the cost of USRP hardware is high. In contrast, low-cost methods often compromise on non-invasive operation. Therefore, a low-cost, non-invasive system may be developed.

Path towards smartphone sensing. Scarf may enable soil carbon to be sensed with smartphones vis smartphone CSI collection and smartphone image calibration. (i) CSI collection on smartphones. CSI is generally unavailable in modern Wi-Fi chips. Earlier CSI extraction tools are limited to outdated chipsets (Daniel Halperin, et al., ACM SIGCOMM computer communication review, 41(1):53-53, 2011., Yaxiong Xie, et al., In Proceedings of the 21st Annual international conference on Mobile Computing and Networking (MobiCom), pages 53-64, 2015.). Nexmon supports more Wi-Fi devices, including smartphones via firmware modification (Matthias Schulz, et al., Nexmon: The c-based firmware patching framework. https://nexmon.org, 2017., Francesco Gringoli, et al., In Proceedings of the 13th International Workshop on Wireless Network Testbeds, Experimental Evaluation & Characterization (WINTECH), page 21-28, 2019.). A recent work, BeamSense, enables CSI extraction using beamforming reports and supports various 802.11-complient Wi-Fi chips without firmware modification (Enze Yi, et al., In 21st USENIX Symposium on Networked Systems Design and Implementation (NSDI), pages 1697-1712, 2024.). Nexmon and BeamSense may be employed for soil carbon sensing. (ii) Image calibration. The quality of soil surface images is affected by the distance and rotation of the smartphone. A system capable of automatically calibrating the positions utilizing a smartphone's internal sensors, e.g., gyroscope and LIDAR may be designed. To account for the impact of lighting conditions and camera models on measured soil lightness, advanced image calibration techniques, e.g., the deep learning-based approach adopted in Sharma et al., may be used (Neha Sharma, et al., In Proceedings of the 29th Annual International Conference on Mobile Computing and Networking (MobiCom), pages 1-15, 2023).

Evaluation for different soil types. Parameters of the models may be adjusted for different soil types. Scarf only considers farms soils without non-carbon color pigments, and high levels of heavy metal and salt contamination. These factors affect soil lightness, permittivity and EC accordingly, and can be handled by updating the mathematical models. To address the issue of limited dataset size and difficulty of real-world data collection, transfer learning methods may be used and may benefit from existing datasets. Transfer Learning is advantageous for small datasets due to its ability to leverage knowledge from a larger dataset to improve the performance of a model trained on a smaller target dataset (Lisa Torrey and Jude Shavlik. In Handbook of re-search on machine learning applications and trends: algorithms, methods, and techniques, pages 242-264. IGI global, 2010.). The ML model trained on one soil type can be adapted to another soil type. The mathematical models of the Example were not sensitive to small soil composition variations (<20%), indicating that models only need to be trained for coarse-grained soil types. The models of the Example may be expanded for larger soil composition variations by adapting model parameters to other soil types.

Improving feature extraction. In this Example, two CSI representations, i.e., CSI image and MUSIC spectrum, were tested. However, any forms of CSI representations may be used. Other CSI representations may result in increased performance. Additionally, the feature extraction network that was adopted may be further improved.

A conclusion of the Example is now described.

Scarf is a novel soil carbon sensing technique that leverages widely available RF signals and images, eliminating the requirement for specialized hardware. To disentangle the complex relationships between soil carbon, soil permittivity, soil moisture, and soil color, mathematical models were derived to compute carbon content from soil permittivity and lightness measured by RF signals and images. Machine learning was combined with the mathematical models to further improve performance. Scarf was tested with field soils and sand compost mixtures at various soil carbon contents and moisture levels. The results showed that Scarf can achieve high accuracy in predicting carbon contents with the combined mathematical and machine learning model. Scarf is a significant step towards a low-cost, rapid and accurate soil carbon sensor that can enable farmers and researchers to monitor soil carbon content and identify site-specific practices of sustainable agriculture.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

The following publications are incorporated herein by reference in their entirety.
Rattan Lal. Soil health and carbon management. Food and Energy Security, 5(4):212-222, 2016.
Rattan Lal. Soils and sustainable agriculture. a review. Agronomy for Sustainable Development, 28:57-64, 2008.
Amitava Chatterjee, Rattan Lal, L Wielopolski, Madhavi Z Martin, and MH Ebinger. Evaluation of different soil carbon determination methods. Critical Reviews in Plant Science, 28(3):164-178, 2009.
Darrell W Nelson and Lee E Sommers. Total carbon, organic carbon, and organic matter. Methods of soil analysis: Part 3 Chemical methods, 5:961-1010, 1996.
The Service Testing and Research Laboratory at Ohio State University. Sample analysis fee schedule. https://bpb-us-w2.wpmucdn.com/u.osu.edu/dist/4/39220/files/2022/07/STAR-lab-analysis-cost_FY-23.pdf, September 2022.
SWPT Laboratory at Colorado State University. Feed and forage analysis price list. https://agsci.colostate.edu/soiltestinglab/price-list/,May 2023.
David A Cremers, Michael H Ebinger, David D Breshears, Pat J Unkefer, Susan A Kammerdiener, Monty J Ferris, Kathryn M Catlett, and Joel RBrown. Measuring total soil carbon with laser-induced breakdown spectroscopy (libs). Journal of Environmental Quality, 30(6):2202-2206, 2001.
Michael H Ebinger, David A Cremers, Clifton M Meyer, Ronny DHarris, et al. Laser-induced breakdown spectroscopy and applications for soil carbon measurement. Carbon sequestration in soils of Latin America. Food Products Press, New York, pages 407-422, 2006.
Lucian Wielopolski, George Hendrey, Kurt H Johnsen, Sudeep Mitra, Stephen A Prior, Hugo H Rogers, and H Allen Torbert. Nondestructive system for analyzing carbon in the soil. Soil Science Society of America Journal, 72(5):1269-1277, 2008.
Skye A Wills, C Lee Burras, and Jonathan A Sandor. Prediction of soil organic carbon content using field and laboratory measurements of soil color. Soil Science Society of America Journal, 71(2):380-388, 2007.
Naoki Moritsuka, Kaori Matsuoka, Keisuke Katsura, Shuji Sano, and Junta Yanai. Soil color analysis for statistically estimating total carbon, total nitrogen and active iron contents in japanese agricultural soils. Soil science and plant nutrition, 60(4):475-485, 2014.
R A Viscarra Rossel, Youssef Fouad, and Christian Walter. Using a digital camera to measure soil organic carbon and iron contents. Biosystems engineering, 100(2):149-159, 2008.\
Luis Gómez-Robledo, Nuria López-Ruiz, Manuel Melgosa, Alberto JPalma, Luis Fermín Capitán-Vallvey, and Manuel Sánchez-Marañón. Using the mobile phone as munsell soil-colour sensor: An experiment under controlled illumination conditions. Computers and electronics in agriculture, 99:200-208, 2013.
Yijia Tang, Edward Jones, and Budiman Minasny. Evaluating low-cost portable near infrared sensors for rapid analysis of soils from southeastern australia. Geoderma Regional, 20: e00240, 2020.
Patrick M Ewing, Dan TerAvest, Xinyi Tu, and Sieglinde S Snapp. Accessible, affordable, fine-scale estimates of soil carbon for sustainable management in sub-saharan africa. Soil Science Society of America Journal, 85(5):1814-1826, 2021.
Amin Sharififar, Kanika Singh, Edward Jones, Frisa Irawan Ginting, and Budiman Minasny. Evaluating a low-cost portable NIR spectrometer for the prediction of soil organic and total carbon using different calibration models. Soil Use and Management, 35(4):607-616, 2019.
Jian Ding and Ranveer Chandra. Towards low cost soil sensing using Wi-Fi. In The 25th Annual International Conference on Mobile Computing and Networking (MobiCom), pages 1-16. ACM, 2019.
Colleen Josephson, Manikanta Kotaru, Keith Winstein, Sachin Katti, and Ranveer Chandra. Low-cost in-ground soil moisture sensing with radar backscatter tags. In ACM SIGCAS Conference on Computing and Sustainable Societies, pages 299-311, 2021.
Zhaoxin Chang, Fusang Zhang, Jie Xiong, Junqi Ma, Beihong Jin, and Daqing Zhang. Sensor-free soil moisture sensing using LoRa signals. Proceedings of the ACM on Interactive, Mobile, Wearable and Ubiquitous Technologies, 6(2):1-27, 2022.
Yuda Feng, Yaxiong Xie, Deepak Ganesan, and Jie Xiong. LTE-Based low-cost and low-power soil moisture sensing. In Proceedings of the 20th ACM Conference on Embedded Networked Sensor Systems (SenSys), pages 421-434, 2022.
Valery Mironov and Igor Savin. A temperature-dependent multi-relaxation spectroscopic dielectric model for thawed and frozen organic soil at 0.05-15 ghz. Physics and Chemistry of the Earth, Parts A/B/C, 83:57-64, 2015.
Chang-Hwan Park, Carsten Montzka, Thomas Jagdhuber, François Jonard, Gabrielle De Lannoy, Jinkyu Hong, Thomas J Jackson, and Volker Wulfmeyer. A dielectric mixing model accounting for soil organic matter. Vadose Zone Journal, 18(1):190036, 2019.
Albert Henry Munsell. A Color Notation: A Measured Color System Based on the Three Qualities, Hue, Values and Chroma with Illustrative Models, Charts and a Course of Study Arranged for Teachers. Geo. H. Ellis, 1907.
Ju Wang, Liqiong Chang, Shourya Aggarwal, Omid Abari, and Srinivasan Keshav. Soil moisture sensing with commodity RFID systems. In Proceedings of the 18th International Conference on Mobile Systems, Applications, and Services (MobiSys), pages 273-285, 2020.
Daniel Kiv, Garvita Allabadi, Berkay Kaplan, and Robin Kravets. smol: Sensing soil moisture using LoRa. In Proceedings of the 1st ACM Workshop on No Power and Low Power Internet-of-Things, pages 21-27, 2022.
Usman Mahmood Khan and Muhammad Shahzad. Estimating soil moisture using RF signals. In Proceedings of the 28th Annual International Conference on Mobile Computing and Networking (MobiCom), pages 242-254, 2022. Rice University. Warp project. warpproject(dot)org
Theodora Angelopoulou, Nikolaos Tziolas, Athanasios Balafoutis, George Zalidis, and Dionysis Bochtis. Remote sensing techniques for soil organic carbon estimation: A review. Remote Sensing, 11(6):676, 2019.
Umesh Acharya, Rattan Lal, and Ranveer Chandra. Data driven approach on in-situ soil carbon measurement. Carbon Management, 13(1):401-419, 2022.
Sidney Allen Bowers. Reflection of radiant energy from soils. Kansas State University, 1971.
Eyal Ben-Dor, Y Inbar, and Y Chen. The reflectance spectra of organic matter in the visible near-infrared and short wave infrared region (400-2500 nm) during a controlled decomposition process. Remote sensing of Environment, 61(1):1-15, 1997.
Marco Nocita, Antoine Stevens, Carole Noon, and Bas van Wesemael. Prediction of soil organic carbon for different levels of soil moisture using vis-nir spectroscopy. Geoderma, 199:37-42, 2013.
Johan Alexander Huisman, Susan Sharpess Hubbard, J David Red-man, and A Peter Annan. Measuring soil water content with ground penetrating radar: A review. Vadose zone journal, 2(4):476-491, 2003.
Kosuke Noborio. Measurement of soil water content and electrical conductivity by time domain reflectometry: a review. Computers and electronics in agriculture, 31(3):213-237, 2001.
Wenjun Jiang, Chenglin Miao, Fenglong Ma, Shuochao Yao, YaqingWang, Ye Yuan, Hongfei Xue, Chen Song, Xin Ma, Dimitrios Koutsonikolas, et al. Towards environment independent device free human activity recognition. In Proceedings of the 24th annual international conference on mobile computing and networking (MobiCom), pages 289-304, 2018.
Yonglong Tian, Guang-He Lee, Hao He, Chen-Yu Hsu, and Dina Katabi. RF-based fall monitoring using convolutional neural networks. Proceedings of the ACM on Interactive, Mobile, Wearable and Ubiquitous Technologies, 2(3):1-24, 2018.
Mingmin Zhao, Yonglong Tian, Hang Zhao, Mohammad Abu Alsheikh, Tianhong Li, Rumen Hristov, Zachary Kabelac, Dina Katabi, and Antonio Torralba. RF-based 3D skeletons. In Proceedings of the 2018 Conference of the ACM Special Interest Group on Data Communication (SIGCOMM), pages 267-281, 2018.
Mingmin Zhao, Tianhong Li, Mohammad Abu Alsheikh, Yonglong Tian, Hang Zhao, Antonio Torralba, and Dina Katabi. Through-wall human pose estimation using radio signals. In Proceedings of the IEEE Conference on Computer Vision and Pattern Recognition (CVPR), pages 7356-7365, 2018.
Shuya Ding, Zhe Chen, Tianyue Zheng, and Jun Luo. RF-net: A unified meta-learning framework for RF-enabled one-shot human activity recognition. In Proceedings of the 18th Conference on Embedded Net-worked Sensor Systems, pages 517-530, 2020.
Tianhong Li, Lijie Fan, Yuan Yuan, and Dina Katabi. Unsupervised learning for human sensing using radio signals. In Proceedings of the IEEE/CVF Winter Conference on Applications of Computer Vision, pages3288-3297, 2022.
Ruiyuan Song, Dongheng Zhang, Zhi Wu, Cong Yu, Chunyang Xie, Shuai Yang, Yang Hu, and Yan Chen. RF-URL: unsupervised representation learning for RF sensing. In Proceedings of the 28th Annual International Conference on Mobile Computing And Networking (Mobi-Com), pages 282-295, 2022.
Kaiming He, Xiangyu Zhang, Shaoqing Ren, and Jian Sun. Deep residual learning for image recognition. In Proceedings of the IEEE conference on computer vision and pattern recognition (CVPR), pages 770-778, 2016.
G W McCarty, J B Reeves, V B Reeves, R F Follett, and J M Kimble. Mid-infrared and near-infrared diffuse reflectance spectroscopy for soil carbon measurement. Soil Science Society of America Journal, 66(2):640-646, 2002.
G Clarke Topp, J L Davis, and Aa P Annan. Electromagnetic determination of soil water content: Measurements in coaxial transmission lines. Water resources research, 16(3):574-582, 1980.
James R Wang and Thomas J Schmugge. An empirical model for the complex dielectric permittivity of soils as a function of water content. IEEE Transactions on Geoscience and Remote Sensing, (4):288-295, 1980.
Myron C Dobson, Fawwaz T Ulaby, Martti T Hallikainen, and Mohamed A El-Rayes. Microwave dielectric behavior of wet soil-part ii: Dielectric mixing models. IEEE Transactions on geoscience and remote sensing, (1):35-46, 1985.
Richard Wayne Newton. Microwave remote sensing and its application to soil moisture detection. Texas A&M University, 1977.
Chang-Hwan Park, Andreas Behrendt, Ellsworth LeDrew, and Volker Wulfmeyer. New approach for calculating the effective dielectric constant of the moist soil for microwaves. Remote Sensing, 9(7):732, 2017.
C H Roth, M A Malicki, and R Plagge. Empirical evaluation of the relationship between soil dielectric constant and volumetric water content as the basis for calibrating soil moisture measurements by tdr. Journal of Soil Science, 43(1):1-13, 1992.
Soil Survey Staff. “web soil survey.” natural resources conservation service, us department of agriculture. 2014.
Deepak Vasisht, Zerina Kapetanovic, Jongho Won, Xinxin Jin, RanveerChandra, Sudipta Sinha, Ashish Kapoor, Madhusudhan Sudarshan, andSean Stratman. {FarmBeats}: an {IoT} platform for {Data-Driven} agriculture. In 14th USENIX Symposium on Networked Systems Design and Implementation (NSDI), pages 515-529, 2017.
Jun Liu, Shaojie Zhao, Lingmei Jiang, Linna Chai, and Fengmin Wu. The influence of organic matter on soil dielectric constant at microwave frequencies (0.5-40 ghz). In 2013 IEEE International Geoscience and Remote Sensing Symposium-IGARSS, pages 13-16. IEEE, 2013.
S K Bhadra and M Bhavanarayana. Estimation of the influence of soil moisture on soil colour. Zeitschrift für Pflanzenernährung und Bodenkunde, 160(3):401-405, 1997.
E A Paul, J A Shields, W K Head, R J St Arnaud, et al. Spectrophotometric measurement of soil color and its relationship to moisture and organic matter.
D F Post, A Fimbres, A D Matthias, E E Sano, L Accioly, A K Batchily, and L G Ferreira. Predicting soil albedo from soil color and spectral reflectance data. Soil Science Society of America Journal, 64(3):1027-1034, 2000.
Manikanta Kotaru, Kiran Joshi, Dinesh Bharadia, and Sachin Katti. SpotFi: Decimeter level localization using wifi. In Proceedings of the 2015 ACM Conference on Special Interest Group on Data Communication (SIGCOMM), pages 269-282, 2015.
Ralph Schmidt. Multiple emitter location and signal parameter estimation. IEEE transactions on antennas and propagation, 34(3):276-280, 1986.
Peter D Hiscocks and P Eng. Measuring luminance with a digital camera. Syscomp electronic design limited, 2, 2011.
Neha Sharma, Muhammad Shahzaib Waseem, Shahrzad Mirzaei, and Mohamed Hefeeda. MobiSpectral: Hyperspectral imaging on mobile devices. In Proceedings of the 29th Annual International Conference on Mobile Computing and Networking (MobiCom), pages 1-15, 2023.
Adam Paszke, Sam Gross, Francisco Massa, Adam Lerer, James Brad-bury, Gregory Chanan, Trevor Killeen, Zeming Lin, Natalia Gimelshein, Luca Antiga, et al. Pytorch: An imperative style, high-performance deep learning library. Advances in neural information processing systems, 32, 2019.
R B Grossman and T G Reinsch. 2.1 bulk density and linear extensibility. Methods of soil analysis: Part 4 physical methods, 5:201-228, 2002.
Arnold Klute. Water retention: laboratory methods. Methods of soil analysis: part 1 physical and mineralogical methods, 5:635-662, 1986.
METER Group. TEROS 10 user manual. 2021.
Daniel Halperin, Wenjun Hu, Anmol Sheth, and David Wetherall. Tool release: Gathering 802.11 n traces with channel state information. ACMSIGCOMM computer communication review, 41(1):53-53, 2011.
Yaxiong Xie, Zhenjiang Li, and Mo Li. Precise power delay profiling with commodity WiFi. In Proceedings of the 21st Annual international conference on Mobile Computing and Networking (MobiCom), pages 53-64, 2015.
Matthias Schulz, Daniel Wegemer, and Matthias Hollick. Nexmon: Thec-based firmware patching framework. https://nexmon.org, 2017.
Francesco Gringoli, Matthias Schulz, Jakob Link, and Matthias Hol-lick. Free your CSI: A channel state information extraction platformfor modern Wi-Fi chipsets. In Proceedings of the 13th International Workshop on Wireless Network Testbeds, Experimental Evaluation & Characterization (WINTECH), page 21-28, 2019.
Enze Yi, Dan Wu, Jie Xiong, Fusang Zhang, Kai Niu, Wenwei Li, and Daqing Zhang. {BFMSense}: {WiFi} sensing using beamforming feed-back matrix. In 21st USENIX Symposium on Networked Systems Design and Implementation (NSDI), pages 1697-1712, 2024. [69] Lisa Torrey and Jude Shavlik. Transfer learning. In Handbook of re-search on machine learning applications and trends: algorithms, methods, and techniques, pages 242-264. IGI global, 2010

What is claimed is:

1. A method for detecting soil carbon content comprising the steps of:
providing an image of a soil surface;
determining soil lightness of the soil surface based on the image;
providing channel state information (CSI) of the soil;
estimating carbon content of the soil based on the soil lightness and the CSI, the estimation being based on a mathematical model;
refining the estimated carbon content, the refinement comprising predicting a first error between the estimated carbon content and the true carbon content using a machine learning model; and
adding the first error to the estimated carbon content.

2. The method of claim 1, wherein the method further comprises the steps of:
estimating the soil water content based on the soil lightness and the CSI, the estimation being based on a mathematical model;
refining the estimated soil water content, the refinement comprising predicting a second error between the estimated soil water content and the true soil water content using a machine learning model; and
adding the second error to the estimated carbon content.

3. The method of claim 1, wherein the machine learning model is trained on images of soil surfaces, soil CSI, and ground truth carbon content.

4. The method of claim 1, wherein the step of estimating the carbon content of the soil comprises determining the effective permittivity of the soil based on the CSI.

5. The method of claim 1, wherein the CSI is determined via assessing the propagation of a radiofrequency (RF) signal in the soil, wherein the RF signal comprises an RF signal selected from the group consisting of: a Wi-Fi signal, a radiofrequency identification (RFID) signal, a backscatter signal, a Radar signal, a LoRa signal, an ultra-wide-band signal, and an LTE signal.

6. The method of claim 1, wherein the image comprises a smartphone image.

7. The method of claim 5, wherein the RF signal is propagated between two RF nodes, at least one of the two RF nodes being buried in the soil.

8. The method of claim 1, wherein the machine learning model comprises a convolutional neural network (CNN).

9. The method of claim 8, wherein the CNN comprises a CNN selected from the group consisting of: ResNet, VGGNet, Inception, DenseNet, MobileNet, and EfficientNet.

10. The method of claim 1, wherein the soil lightness relates to a lightness value in CIELAB color space.

11. The method of claim 1, wherein the soil lightness is normalized based on a parameter selected from the group consisting of: scene luminance, exposure time, aperture size, ISO, camera make, and camera model.

12. The method of claim 1, wherein the soil comprises a field soil or a sand compost mixture.

13. A system for determining soil carbon content, comprising:
two radiofrequency (RF) nodes, the RF nodes being positioned such that the soil is between the nodes;
a camera;
a processor communicatively connected to the RF nodes and the camera; and
a non-transitory computer readable medium with instructions stored thereon, which when executed by a processor perform steps comprising:
acquiring an image of the soil surface via the camera;
determining soil lightness of the soil surface based on the image;
propagating a radiofrequency (RF) signal between the two RF nodes;
determining the CSI of the soil based on assessing the propagation of the RF signal between the nodes;
estimating carbon content of the soil based on the soil lightness and the CSI, the estimation being based on a mathematical model;
refining the estimated carbon content, the refinement comprising predicting a first error between the estimated carbon content and the true carbon content using a machine learning model; and
adding the first error to the estimated carbon content.

14. The system of claim 13, further comprising the steps of
estimating the soil water content based on the soil lightness and the CSI, the estimation being based on a mathematical model;
refining the estimated soil water content, the refinement comprising predicting a second error between the estimated soil water content and the true soil water content using a machine learning model; and
adding the second error to the estimated carbon content.

15. The system of claim 13, wherein the machine learning model is trained on images of soil surfaces, soil CSI, and ground truth carbon content.

16. The system of claim 13, wherein the RF signal comprises an RF signal selected from the group consisting of: a Wi-Fi signal, an RFID signal, a backscatter signal, a Radar signal, a LoRa signal, an ultra-wide-band signal, and an LTE signal.

17. The system of claim 13, wherein the camera comprises a smartphone camera.

18. The system of claim 13, wherein at least one of the two RF nodes is buried in the soil.

19. The system of claim 13, wherein the two RF nodes are connected to a WARP board or a Wi-Fi chipset.

20. The system of claim 13, wherein the soil comprises a field soil or a sand compost mixture.
